(19) United States
(12) Patent Application Publication
(10) Pub. No.: US 2020/0246275 A1
(43) Pub. Date: Aug. 6, 2020

(54) COMBINATION THERAPY COMPRISING NANOPARTICLES OF A TAXANE AND ALBUMIN WITH ABT-263 IN METHODS FOR TREATING CANCER

(71) Applicant: Abraxis BioScience, LLC, Summit, NJ (US)

(72) Inventors: Daniel PIERCE, Belmont, CA (US); Michael CARLETON, Churchville, PA (US)

(21) Appl. No.: 16/678,980

(22) Filed: Nov. 8, 2019

Related U.S. Application Data
(63) Continuation of application No. 15/302,134, filed on Oct. 5, 2016, now abandoned.
(60) Provisional application No. 62/085,020, filed on Nov. 26, 2014.
(60) Provisional application No. 61/975,890, filed on Apr. 6, 2014.

(51) Int. Cl. A61K 9/51; A61K 31/337; A61K 31/5377; A61K 9/00; A61K 31/282; A61K 31/7068

(52) U.S. Cl. CPC A61K 9/5169; A61K 31/337; A61K 31/5377; A61K 31/7068; A61K 9/0053; A61K 31/282; A61K 9/0019

(57) ABSTRACT

Provided are methods of treating a cancer (such as lung cancer, breast cancer, pancreatic cancer, etc.) in an individual in need thereof, comprising administering to the individual a) an effective amount of a composition comprising nanoparticles comprising a taxane (such as paclitaxel) and an albumin (such as human serum albumin), and b) an effective amount of ABT-263.

### CROSS-REFERENCE TO RELATED APPLICATIONS

This application claims the priority benefit of U.S. Provisional Patent Application No. 62/085,020, filed Nov. 26, 2014, and U.S. Provisional Patent Application No. 61/975,890, filed Apr. 6, 2014, the disclosures of which are incorporated by reference in their entireties for all purposes.

### TECHNICAL FIELD

The present invention relates to methods and compositions for the treatment of cancer comprising the administration of a combination of an albumin-based nanoparticle form of a taxane and a second agent.

### BACKGROUND

Cancer is a leading cause of death worldwide, and many cancers are difficult to treat. For example, pancreatic cancer has one of the highest mortality rates among all cancers and is expected to cause an estimated 39,590 deaths in the United States in 2014. See American Cancer Society, Cancer Facts and Figures 2014. For all stages of pancreatic cancer combined, the 1- and 5-year relative survival rates are 27% and 6%, respectively; this high mortality rate from pancreatic cancer is, at least in part, due to the high incidence of metastatic disease at the time of diagnosis. See id. As a result, treatment options for pancreatic cancer are very limited. Similarly, lung cancer is the leading cause of cancer death in both men and women in the United States. In 2013, an estimated 228,190 new cases were diagnosed, and about 159,480 deaths resulted from this disease. More women die from lung cancer than breast, ovarian, and uterine cancer combined, and 3 times as many men die from lung cancer than from prostate cancer. Most patients who are diagnosed with NSCLC cannot be cured with surgery and will eventually die from their disease. See Howlader et al. (eds). SEER Cancer Statistics Review, 1975-2010, National Cancer Institute. Bethesda, Md., based on November 2012 SEER data submission, posted to the SEER web site, April 2013. The median survival of patients with untreated metastatic NSCLC is only four to five months with a survival rate at one year of only 10 percent. Rapp E. et al. J Clin Oncol. 1988; 6:633-41.

Albumin-based nanoparticle compositions have been developed as a drug delivery system for delivering substantially water insoluble drugs such as taxanes. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer as well as pancreatic cancer in the United States.

ABT-263 has been described in U.S. Patent Application 2007/0027135 and U.S. Pat. No. 8,362,013.

The disclosures of all publications, patents, patent applications and published patent applications referred to herein are hereby incorporated herein by reference in their entirety.

### BRIEF SUMMARY OF THE INVENTION

The invention provides combination therapy methods of treating a cancer, comprising administering to the individual a) an effective amount of a composition comprising nanoparticles comprising a taxane (such as paclitaxel) and an albumin, and b) an effective amount of ABT-263. In some embodiments, the invention provides a method of treating a cancer in an individual comprising administering to the individual a) an effective amount of a composition comprising nanoparticles comprising paclitaxel coated with an albumin (such as nab-paclitaxel for example Abraxane®), and b) an effective amount of ABT-263. In some embodiments, the invention provides a method of treating a cancer in an individual comprising administering to the individual a) an effective amount of a composition comprising nanoparticles comprising paclitaxel coated with an albumin (such as nab-paclitaxel for example Abraxane®), wherein the nanoparticle composition is administered at the dosage range of about 60 to about 300 mg/m² (including about 80 to about 200 mg/m², for example about 80 to about 150 mg/m² or about 80 to about 125 mg/m²) and b) an effective amount of ABT-263, wherein the ABT-263 is administered at the dosage range of about 10 to about 300 mg/day (including about 50 to about 200 mg/day, for example about 100 to about 200 mg/day). In some embodiments, the invention provides a method of treating a cancer in an individual comprising administering to the individual a) an effective amount of a composition comprising nanoparticles comprising paclitaxel coated with an albumin (such as nab-paclitaxel for example Abraxane®), wherein the nanoparticle composition is administered intravenously at the dosage range of about 60 to about 300 mg/m² (including about 80 to about 200 mg/m², for example about 80 to about 150 mg/m² or about 80 to about 125 mg/m²) and b) an effective amount of ABT-263, wherein the ABT-263 is administered orally at the dosage range of about 10 to about 300 mg/day (including about 50 to about 200 mg/day, for example about 100 to about 200 mg/day).

In some embodiments, the cancer is resistant or refractory to the treatment of taxane when administered alone or in conjunction with an agent other than ABT-263. In some embodiments, the cancer is resistant or refractory to the treatment when ABT-263 is administered alone or in conjunction with an agent other than the nanoparticle composition (such as a non-nanoparticle composition of a taxane including paclitaxel). In some embodiments, the individual is selected for treatment based on a high level of Bcl-xL. In some embodiments, the individual is selected for treatment based on a low level of Bcl-xL. In some embodiments, the individual is selected for treatment based on a high level of Bcl-2. In some embodiments, the individual is selected for treatment based on a low level of Bcl-2. In some embodiments, the individual is selected for treatment based on a high level of Bcl-xL and Bcl-2. In some embodiments, the individual is selected for treatment based on a high level of Bcl-xL and/or Bcl-2, and a low level of Mcl-1. In some embodiments, the individual is selected for treatment based on the presence of functional caspase 3 and/or caspase 8.

In some embodiments, the composition comprising nanoparticles (also referred to as “nanoparticle composition”) and the ABT-263 are administered simultaneously, either in the same composition or in separate compositions. In some embodiments, the nanoparticle composition and the ABT-263 are administered sequentially, i.e., the nanoparticle composition is administered either prior to or after the administration of the ABT-263.

In some embodiments, the administration of the nanoparticle composition and the ABT-263 is concurrent, i.e., the administration period of the nanoparticle composition and that of the ABT-263 overlap with each other. In some embodiments, the nanoparticle composition is administered for at least one cycle (for example, at least any of 2, 3, or 4 cycles) prior to the administration of the ABT-263. In some embodiments, the ABT-263 is administered for at least any of one, two, three, or four weeks after the termination of the nanoparticle composition. In some embodiments, the nanoparticle composition and the ABT-263 are administered over the same treatment cycles. For example, in some embodiments, the nanoparticle composition is administered weekly in a 21 day cycle and the ABT-263 is administered on days 1, 2, 3, and/or 4 of the first four days of the 21-day cycle. In some embodiments, the nanoparticle composition is administered weekly, three out of four weeks in a 28 day cycle, and the ABT-263 is administered on days 1, 2, 3, and/or 4 of the first four days of the 28-day cycle.

In some embodiments, the cancer is selected from the group consisting of lung cancer, pancreatic cancer, breast cancer, gastric cancer, colorectal cancer, renal cancer, bladder cancer, ovarian cancer, prostate cancer, leukemia, lymphoma, and multiple myeloma. In some embodiments, the cancer is squamous cell carcinoma. In some embodiments, the cancer is adenocarcinoma. In some embodiments, the cancer is characterized by high Bcl-xL expression. In some embodiments, the cancer is characterized by low Bcl-xL expression. In some embodiments, the cancer is characterized by high Bcl-xL expression and low Bcl-2 expression.

Thus, for example, in some embodiments, there is provided a method of treating lung cancer (such as NSCLC), comprising administering to the individual a) an effective amount of a composition comprising nanoparticles comprising a taxane (such as paclitaxel) and an albumin, and b) an effective amount of ABT-263. In some embodiments, the invention provides a method of treating a lung cancer (such as NSCLC) in an individual comprising administering to the individual a) an effective amount of a composition comprising nanoparticles comprising paclitaxel coated with an albumin (such as nab-paclitaxel for example Abraxane®), and b) an effective amount of ABT-263. In some embodiments, the invention provides a method of treating a lung cancer (such as NSCLC) in an individual comprising administering to the individual a) an effective amount of a composition comprising nanoparticles comprising paclitaxel coated with an albumin (such as nab-paclitaxel for example Abraxane®), wherein the nanoparticle composition is administered at the dosage range of about 60 to about 300 mg/m² (including about 80 to about 200 mg/m², for example about 80 to about 150 mg/m² or about 80 to about 125 mg/m²) and b) an effective amount of ABT-263, wherein the ABT-263 is administered at the dosage range of about 10 to about 300 mg/day (including about 50 to about 200 mg/day, for example about 100 to about 200 mg/day). In some embodiments, the invention provides a method of treating a lung cancer (such as NSCLC) in an individual comprising administering to the individual a) an effective amount of a composition comprising nanoparticles comprising paclitaxel coated with an albumin (such as nab-paclitaxel for example Abraxane®), wherein the nanoparticle composition is administered intravenously at the dosage range of about 60 to about 300 mg/m² (including about 80 to about 200 mg/m², for example about 80 to about 150 mg/m² or about 80 to about 125 mg/m²) and b) an effective amount of ABT-263, wherein the ABT-263 is administered orally at the dosage range of about 10 to about 300 mg/day (including about 50 to about 200 mg/day, for example about 100 to about 200 mg/day). In some embodiments, according to any one of the lung cancer treatment method described above, the method further comprises administering to the individual an effective amount of carboplatin. In some embodiments, the carboplatin is administered (for example intravenously administered) at the dosage of AUC=2 to AUC=6 (such as AUC=6). In some embodiments according to any one of the lung cancer treatment methods described above, the lung cancer is squamous cell carcinoma (such as squamous NSCLC). In some embodiments, the lung cancer is adenocarcinoma (such as adenocarcinoma NSCLC).

In some embodiments, there is provided a method of treating pancreatic cancer (such as metastatic pancreatic cancer or locally advanced pancreatic cancer), comprising administering to the individual a) an effective amount of a composition comprising nanoparticles comprising a taxane (such as paclitaxel) and an albumin, and b) an effective amount of ABT-263. In some embodiments, the invention provides a method of treating a pancreatic cancer (such as metastatic pancreatic cancer or locally advanced pancreatic cancer) in an individual comprising administering to the individual a) an effective amount of a composition comprising nanoparticles comprising paclitaxel coated with an albumin (such as nab-paclitaxel for example Abraxane®), and b) an effective amount of ABT-263. In some embodiments, the invention provides a method of treating a pancreatic cancer (such as metastatic pancreatic cancer or locally advanced pancreatic cancer) in an individual comprising administering to the individual a) an effective amount of a composition comprising nanoparticles comprising paclitaxel coated with an albumin (such as nab-paclitaxel for example Abraxane®), wherein the nanoparticle composition is administered at the dosage range of about 60 to about 300 mg/m² (including about 80 to about 200 mg/m², for example about 80 to about 150 mg/m² or about 80 to about 125 mg/m²) and b) an effective amount of ABT-263, wherein the ABT-263 is administered at the dosage range of about 10 to about 300 mg/day (including about 50 to about 200 mg/day, for example about 100 to about 200 mg/day). In some embodiments, the invention provides a method of treating a pancreatic in an individual comprising administering to the individual a) an effective amount of a composition comprising nanoparticles comprising paclitaxel coated with an albumin (such as nab-paclitaxel for example Abraxane®), wherein the nanoparticle composition is administered intravenously at the dosage range of about 60 to about 300 mg/m² (including about 80 to about 200 mg/m², for example about 80 to about 150 mg/m² or about 80 to about 125 mg/m²) and b) an effective amount of ABT-263, wherein the ABT-263 is administered orally at the dosage range of about 10 to about 300 mg/day (including about 50 to about 200 mg/day, for example about 100 to about 200 mg/day). In some embodiments, according to any one of the pancreatic cancer treatment method described above, the method further comprises administering to the individual an effective amount of gemcitabine. In some embodiments, the gemcitabine is administered (for example intravenously administered) at the dosage of about 1000 to about 2000 mg/m². In some embodiments according to any one of the pancreatic cancer treatment methods described above, the pancreatic cancer is squamous cell carcinoma (such as squamous pancreatic cancer). In some embodiments, the pancreatic cancer is adenocarcinoma (such as adenocarcinoma pancreatic cancer).

In some embodiments, there is provided a method of treating breast cancer in an individual, comprising administering to the individual a) an effective amount of a composition comprising nanoparticles comprising a taxane (such as paclitaxel) and an albumin, and b) an effective amount of ABT-263. In some embodiments, there is provided a method of treating breast cancer in an individual, wherein the individual is negative for ER, PR, and HER2, comprising administering to the individual a) an effective amount of a composition comprising nanoparticles comprising a taxane (such as paclitaxel) and an albumin, and b) an effective amount of ABT-263. In some embodiments, the method further comprises conducting definitive surgery within about 1, 2, 3, 4, 5, 6, 7, 8, 9, or 10 days following the preoperative therapy.

The methods of the invention generally comprise administration of a composition comprising nanoparticles comprising a taxane (such as paclitaxel) and an albumin. In some embodiments, the nanoparticle composition comprises nanoparticles comprising paclitaxel coated with an albumin. In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of all the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of all the nanoparticles in the composition fall within the range of about 20 to about 400, including for example about 20 to about 200 nm, about 30 to about 180 nm, and any one of about 40 to about 150, about 50 to about 120, and about 60 to about 100 nm.

In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the composition comprises taxane in both nanoparticle and non-nanoparticle form, wherein at least about any one of 50%, 60%, 70%, 80%, 90%, 95%, or 99% of the taxane in the composition are in nanoparticle form. In some embodiments, the taxane in the nanoparticles constitutes more than about any one of 50%, 60%, 70%, 80%, 90%, 95%, or 99% of the nanoparticles by weight.

In some embodiments, the nanoparticle composition is substantially free (such as free) of surfactants (such as Cremophor®, Tween 80, or other organic solvents used for the administration of taxanes). In some embodiments, the nanoparticle composition contains less than about any one of 20%, 15%, 10%, 7.5%, 5%, 2.5%, or 1% organic solvent.

In some embodiments, the weight ratio of albumin and taxane in the nanoparticle composition is about 18:1 or less, such as about 15:1 or less, for example about 9:1 or less. In some embodiments, the weight ratio of albumin and taxane in the composition falls within the range of any one of about 1:1 to about 18:1, about 2:1 to about 15:1, about 3:1 to about 13:1, about 4:1 to about 12:1, about 5:1 to about 10:1, about 9:1 (for example about 1:1 to about 9:1). In some embodiments, the weight ratio of taxane and albumin in the nanoparticle portion of the composition is about any one of 1:2, 1:3, 1:4, 1:5, 1:9, 1:10, 1:15, or less.

In some embodiments, the particle composition comprises one or more of the above characteristics.

In some embodiments, the nanoparticle composition is Abraxane® (nab-paclitaxel). Nanoparticle compositions comprising other taxanes (such as docetaxel and ortataxel) may also comprise one or more of the above characteristics.

Also provided are kits and compositions useful for methods described herein.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF FIGURES

FIG. 1A shows the effect of Abraxane® on CC3 and pHH3 expression in MiaPACA2 cells. FIG. 1B shows the effect of ABT-263 on CC3 and pHH3 expression in MiaPACA2 cells. FIG. 1C shows the effect of Abraxane® in combination with ABT-263 on CC3 and pHH3 expression in MiaPACA2 cells. FIG. 1D shows the effect of ABT-199 on CC3 and pHH3 expression in MiaPACA2 cells. FIG. 1E shows the effect of Abraxane® in combination with ABT-263 on CC3 and pHH3 expression in MiaPACA2 cells.

FIG. 2A shows percent CC3 positive cells plotted as a function of radial distance in MiaPACA2 tumors injected with Abraxane® alone, ABT-199 alone, Abraxane® in combination with ABT-199, or vehicle. FIG. 2B shows percent CC3 positive cells plotted as a function of radial distance in MiaPACA2 tumors injected with Abraxane® alone, ABT-263 alone, Abraxane® in combination with ABT-263, or vehicle.

FIG. 3A shows the effect of Abraxane® alone, ABT-263 alone, Abraxane® in combination with ABT-263, and vehicle on CC3 expression in MiaPACA2 cells after 24 hours. FIG. 3B shows the effect of Abraxane® alone, ABT-263 alone, Abraxane® in combination with ABT-263, and vehicle on CC3 expression in MiaPACA2 cells after 48 hours.

FIG. 4A shows the effect of Abraxane® alone, ABT-263 alone, Abraxane® in combination with ABT-263, and vehicle on pHH3 expression in MiaPACA2 cells after 24 hours. FIG. 4B shows the effect of Abraxane® alone, ABT-263 alone, Abraxane® in combination with ABT-263, and vehicle on pHH3 expression in MiaPACA2 cells after 48 hours.

FIG. 5 shows mean values of percent CC3 positive cells plotted with standard error bars, as a function of radial distance for Abraxane®+ABT-263.

FIG. 6A shows the expression of Bcl-2 in MiaPACA2 cells. FIG. 6B shows the expression of Bcl-xL in MiaPACA2 cells.

FIG. 7A shows the effect of Abraxane® in combination with gemcitabine on CC3 and pHH3 expression in MiaPACA2 cells. FIG. 7B shows the effect of Abraxane® in combination with ABT-263 on CC3 and pHH3 expression in MiaPACA2 cells. FIG. 7C shows percent CC3 positive cells plotted as a function of radial distance in MiaPACA2 tumors injected with Abraxane® in combination with ABT-263 or Abraxane® in combination with gemcitabine.

FIG. 8A shows the effect of Abraxane® on CC3 and pHH3 expression in H2122 cells. FIG. 8B shows the effect of ABT-263 on CC3 and pHH3 expression in H2122 cells. FIG. 8C shows the effect of Abraxane® in combination with ABT-263 on CC3 and pHH3 expression in H2122 cells.

FIG. 9 shows percent CC3 positive cells plotted as a function of radial distance in H2122 tumors injected with Abraxane® alone, ABT-263 alone, or Abraxane® in combination with ABT-263.

FIG. 10A shows the expression of Bcl-2 in H2122 cells. FIG. 10B shows the expression of Bcl-xL in H2122 cells.

### DETAILED DESCRIPTION OF THE INVENTION

The present invention provides methods of combination therapy comprising administration of nanoparticles comprising a taxane (such as paclitaxel) and an albumin in conjunction with ABT-263. The present invention is based on the striking discovery that Abraxane®, an albumin bound nanoparticle formulation of paclitaxel, showed significant synergy with ABT-263 in various solid tumor models in inducing tumor cell death. Combination therapy of a nanoparticle composition comprising a taxane (such as paclitaxel) and an albumin with ABT-263 at effective dosages would therefore significantly improve the efficacy of nanoparticle forms of taxane-based therapy and/or the efficacy of ABT-263 in cancer treatment. Such method may lead to better efficacy with reduced, or manageable, toxicity (such as hematological toxicity).

Notably, the synergistic effect discussed above was not observed with ABT-199, a specific Bcl-2 inhibitor, suggesting that the effects of ABT-263 may be attributable to its ability to inhibit Bcl-xL independently of, or in addition to Bcl-2. The present application thus provides combination methods for treating cancer with a nanoparticle composition comprising a taxane (such as paclitaxel) and an albumin and a Bcl-xL inhibitor (such as a Bcl-xL/Bcl-2 dual inhibitor or a Bcl-xL inhibitor that does not have Bcl-2 inhibitory activity). While the present application focuses primarily on ABT-263, it is to be understood that discussion on ABT-263 throughout the present application are equally applicable to other Bcl-xL inhibitors (such as a Bcl-xL/Bcl-2 dual inhibitor or a Bcl-xL inhibitor that does not have Bcl-2 inhibitory activity).

The present application thus provides methods of combination therapy. It is to be understood by a person of ordinary skill in the art that the combination therapy methods described herein requires that the nanoparticle composition be administered in conjunction with an ABT-263. “In conjunction with” refers to administration of one treatment modality in addition to another treatment modality, such as administration of a nanoparticle composition described herein in addition to administration of the ABT-263 to the same individual. As such, “in conjunction with” refers to administration of one treatment modality before, during or after delivery of the other treatment modality to the individual.

The methods described herein are generally useful for treatment of cancers. As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, and remission (whether partial or total). Also encompassed by “treatment” is a reduction of pathological consequence of a proliferative disease. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation.

The term “individual” is a mammal, including humans. An individual includes, but is not limited to, human, bovine, horse, feline, canine, rodent, or primate. In some embodiments, the individual is human.

The methods may be practiced in an adjuvant setting. “Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

The methods provided herein may also be practiced in a “neoadjuvant setting,” i.e., the method may be carried out before the primary/definitive therapy. In some embodiments, the individual has previously been treated. In some embodiments, the individual has not previously been treated. In some embodiments, the treatment is a first line therapy.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Methods of Combination Therapy

The present invention provides methods of treating cancer in an individual, comprising administering to the individual: a) an effective amount of a composition comprising nanoparticles comprising a taxane (such as paclitaxel) and an albumin; and b) an effective amount of a Bcl-xL inhibitor. In some embodiments, there is provided a method of treating cancer in an individual, comprising administering to the individual: a) an effective amount of a composition comprising nanoparticles comprising a taxane (such as paclitaxel) and an albumin; and b) an effective amount of a dual Bcl-xL/Bcl-2 inhibitor. In some embodiments, there is provided a method of treating cancer in an individual, comprising administering to the individual: a) an effective amount of a composition comprising nanoparticles comprising a taxane (such as paclitaxel) and an albumin; and b) an effective amount of a Bcl-xL inhibitor that does not have Bcl-2 inhibitory activity (for example a BH3 mimetic having Bcl-xL inhibitory activity but not Bcl-2 inhibitory activity). In some embodiments, the nanoparticle composition and the Bcl-xL inhibitor are administered simultaneously. In some embodiments, the nanoparticle composition and the Bcl-xL are administered sequentially.

The present invention provides methods of treating cancer in an individual, comprising administering to the individual: a) an effective amount of a composition comprising nanoparticles comprising a taxane (such as paclitaxel) and an albumin; and b) an effective amount of ABT-263. In some embodiments, the nanoparticle composition and the ABT-263 are administered simultaneously. In some embodiments, the nanoparticle composition and the ABT-263 are administered sequentially.

In some embodiments, there is provided a method of treating cancer in an individual, comprising administering to the individual: a) an effective amount of a composition comprising nanoparticles comprising a taxane (such as paclitaxel) and an albumin; and b) an effective amount of ABT-263, wherein the nanoparticle composition and the ABT-263 are administered concurrently. In some embodiments, the administrations of the nanoparticle composition and the ABT-263 are initiated at about the same time (for example, within any one of 1, 2, 3, 4, 5, 6, or 7 days). In some embodiments, the administrations of the nanoparticle composition and the ABT-263 are terminated at about the same time (for example, within any one of 1, 2, 3, 4, 5, 6, or 7 days). In some embodiments, the administration of the ABT-263 continues (for example for about any one of 0.5, 1, 2, 3, 4, 5, 6, 7, 8, 9, 10, 11, or 12 months) after the termination of the administration of the nanoparticle composition. In some embodiments, the administration of the ABT-263 continues (for example for about any one of 0.5, 1, 2, 3, 4, 5, 6, 7, 8, 9, 10, 11, 12, 13, or 14 days) after the termination of the administration of the nanoparticle composition. In some embodiments, the administration of the ABT-263 is initiated after (for example after about any one of 0.5, 1, 2, 3, 4, 5, 6, 7, 8, 9, 10, 11, or 12 months) the initiation of the administration of the nanoparticle composition. In some embodiments, the administration of the ABT-263 is initiated after (for example after about any one of 0.5, 1, 2, 3, 4, 5, 6, 7, 8, 9, 10, 11, 12 13 or 14 days) the initiation of the administration of the nanoparticle composition. In some embodiments, the administrations of the nanoparticle composition and the ABT-263 are initiated and terminated at about the same time. In some embodiments, the administrations of the nanoparticle composition and the ABT-263 are initiated at about the same time and the administration of the ABT-263 continues (for example for about any one of 0.5, 1, 2, 3, 4, 5, 6, 7, 8, 9, 10, 11, or 12 months) after the termination of the administration of the nanoparticle composition. In some embodiments, the administrations of the nanoparticle composition and the ABT-263 are initiated at about the same time and the administration of the ABT-263 continues (for example for about any one of 0.5, 1, 2, 3, 4, 5, 6, 7, 8, 9, 10, 11, 12, 13 or 14 days) after the termination of the administration of the nanoparticle composition. In some embodiments, the administration of the nanoparticle composition and the ABT-263 stop at about the same time and the administration of the ABT-263 is initiated after (for example after about any one of 0.5, 1, 2, 3, 4, 5, 6, 7, 8, 9, 10, 11, or 12 months) the initiation of the administration of the nanoparticle composition. In some embodiments, the administration of the nanoparticle composition and the ABT-263 stop at about the same time and the administration of the ABT-263 is initiated after (for example after about any one of 0.5, 1, 2, 3, 4, 5, 6, 7, 8, 9, 10, 11, 12, 13 or 14 days) the initiation of the administration of the nanoparticle composition. In some embodiments, the administration of the nanoparticle composition and the ABT-263 stop at about the same time and the administration of the nanoparticle composition is initiated after (for example after about any one of 0.5, 1, 2, 3, 4, 5, 6, 7, 8, 9, 10, 11, or 12 months) the initiation of the administration of the ABT-263. In some embodiments, the administration of the nanoparticle composition and the ABT-263 stop at about the same time and the administration of the nanoparticle composition is initiated after (for example after about any one of 0.5, 1, 2, 3, 4, 5, 6, 7, 8, 9, 10, 11, 12, 13, or 14 days) the initiation of the administration of the ABT-263.

In some embodiments, the taxane is any of (and in some embodiments consisting essentially of) paclitaxel, docetaxel, tesetaxel, and ortataxel. In some embodiments, the taxane is paclitaxel. In some embodiments, the taxane is docetaxel. In some embodiments, the nanoparticle composition comprises nab-paclitaxel (such as Abraxane®). In some embodiments, the nanoparticle composition is nab-paclitaxel (such as Abraxane®).

Thus, for example, in some embodiments, there is provided a method of treating cancer in an individual, comprising administering to the individual: a) an effective amount of a composition comprising nanoparticles comprising a taxane (such as paclitaxel) coated with an albumin; and b) an effective amount of ABT-263. In some embodiments, the nanoparticles have an average size of about 20 to about 400 nm, such as about 40 to about 200 nm. In some embodiments, there is provided a method of treating cancer in an individual, comprising administering to the individual: a) an effective amount nab-paclitaxel (such as Abraxane®); and b) an effective amount of ABT-263. In some embodiments, the nanoparticle composition and the ABT-263 are administered simultaneously. In some embodiments, the nanoparticle composition and the ABT-263 are administered sequentially. In some embodiments, the proliferative disease is a cancer selected from the group consisting of breast cancer, lung cancer (such as small cell lung cancer and non-small cell lung cancer), renal cancer, bladder cancer, pancreatic cancer, ovarian cancer, prostate cancer, gastric cancer, colorectal cancer, leukemia, lymphoma, and multiple myeloma.

In some embodiments, there is provided a method of treating cancer in an individual, comprising administering to the individual: a) an effective amount of a composition comprising nanoparticles comprising a taxane (such as paclitaxel) and an albumin; and b) an effective amount of ABT-263, wherein the nanoparticle composition is administered intravenously and wherein the ABT-263 is administered orally. In some embodiments, there is provided a method of treating cancer in an individual, comprising administering to the individual: a) an effective amount of a composition comprising nanoparticles comprising paclitaxel coated with an albumin (such as nab-paclitaxel, for example Abraxane); and b) an effective amount of ABT-263, wherein the nanoparticle composition is administered intravenously and wherein the ABT-263 is administered orally. In some embodiments, the nanoparticle composition and the ABT-263 are administered sequentially. In some embodiments, the proliferative disease is a cancer selected from the group consisting of breast cancer, lung cancer (such as small cell lung cancer and non-small cell lung cancer), renal cancer, bladder cancer, pancreatic cancer, ovarian cancer, prostate cancer, gastric cancer, colorectal cancer, leukemia, lymphoma, and multiple myeloma.

In some embodiments, there is provided a method of treating cancer in an individual, comprising administering to the individual: a) an effective amount of a composition comprising nanoparticles comprising a taxane (such as paclitaxel) and an albumin, wherein the nanoparticle composition is in the dosage range of about 60-300 mg/m² (including for example about 80 to about 200 mg/m², for example about 80 to about 150 mg/m² or about 80 to about 125 mg/m²), and b) about 10 to about 300 mg/day (including about 50 to about 200 mg/day, for example about 100 to about 200 mg/day) ABT-263. In some embodiments, there is provided a method of treating cancer in an individual, comprising administering to the individual: a) an effective amount of a composition comprising nanoparticles comprising paclitaxel coated with an albumin (such as nab-paclitaxel for example Abraxane®), wherein the taxane is in the dosage range of about 60-300 mg/m² (including for example about 80 to about 200 mg/m², for example about 80 to about 150 mg/m² or about 80 to about 125 mg/m²), and b) about 10 to about 300 mg/day (including about 50 to about 200 mg/day, for example about 100 to about 200 mg/day) ABT-263. In some embodiments, the nanoparticle composition is administered intravenously. In some embodiments, the ABT-263 is administered orally. In some embodiments, the administration of ABT-263 is synchronized with at least one dose of nanoparticle composition. In some embodiments, the nanoparticle composition is administered on a two, three, or four week dosing cycle, and the ABT-263 is administered on any 1, 2, 3, or 4 days of the first four days of the dosing cycle. In some embodiments, the nanoparticle composition is administered two out of three weeks on a three week cycle (for example on days 1 and 8 of a three week cycle). In some embodiments, the nanoparticle composition is administered three out of four weeks on a four week cycle (for example on days 1, 8, and 15 of a four week cycle). In some embodiments the nanoparticle composition is administered two out of four weeks on a four week cycle (for example on days 1 and 15 of a four week cycle). In some embodiments, the nanoparticle composition is administered weekly on a four week cycle (for example on days 1, 8, 15, and 21 of a four-week cycle). In some embodiments, the nanoparticle composition is administered weekly on a three week cycle (for example on days 1, 8, and 15 of a three-week cycle).

In some embodiments, there is provided a method of treating cancer in an individual, comprising administering to the individual: a) an effective amount of a composition comprising nanoparticles comprising a taxane (such as paclitaxel) and an albumin, wherein the nanoparticle composition is administered intravenously in the dosage range of about 60-300 mg/m² (including for example about 80 to about 200 mg/m², for example about 80 to about 150 mg/m² or about 80 to about 125 mg/m²), and b) an effective amount of ABT-263, wherein the ABT-263 is administered orally at the dosage of about 10 to about 300 mg/day (including about 50 to about 200 mg/day, for example about 100 to about 200 mg/day). In some embodiments, there is provided a method of treating cancer in an individual, comprising administering to the individual: a) an effective amount of a composition comprising nanoparticles comprising paclitaxel coated with an albumin (such as nab-paclitaxel for example Abraxane®), wherein the nanoparticle composition is administered intravenously in the dosage range of about 60-300 mg/m² (including for example about 80 to about 200 mg/m², for example about 80 to about 150 mg/m² or about 80 to about 125 mg/m²), and b) an effective amount of ABT-263, wherein the ABT-263 is administered orally at the dosage of about 10 to about 300 mg/day (including about 50 to about 200 mg/day, for example about 100 to about 200 mg/day). In some embodiments, there is provided a method of treating cancer in an individual, comprising administering to the individual: a) an effective amount of a composition comprising nanoparticles comprising paclitaxel coated with an albumin (such as nab-paclitaxel for example Abraxane®), wherein the taxane is in the dosage range of about 80 to about 125 mg/m² (such as any of about 80, 100, or 125 mg/m²) and b) about 100 to about 200 mg/day ABT-263. In some embodiments, the nanoparticle composition is administered first followed by administration of the ABT-263. In some embodiments, the ABT-263 is administered first followed by administration of the nanoparticle composition. In some embodiments, the administration of ABT-263 is synchronized with at least one dose of nanoparticle composition. In some embodiments, the nanoparticle composition is administered on a two, three, or four week dosing cycle, and the ABT-263 is administered 1, 2, 3, or 4 days out of the first four days of the dosing cycle. In some embodiments, the nanoparticle composition is administered two out of three weeks on a three week cycle (for example on days 1 and 8 of a three week cycle). In some embodiments, the nanoparticle composition is administered three out of four weeks on a four week cycle (for example on days 1, 8, and 15 of a four week cycle). In some embodiments the nanoparticle composition is administered two out of four weeks on a four week cycle (for example on days 1 and 15 of a four week cycle). In some embodiments, the nanoparticle composition is administered weekly on a four week cycle (for example on days 1, 8, 15, and 21 of a four-week cycle). In some embodiments, the nanoparticle composition is administered weekly on a three week cycle (for example on days 1, 8, and 15 of a three-week cycle).

The methods described herein are suitable for treating various cancers, such as cancers described herein, including a cancer selected from the group consisting of lung cancer, pancreatic cancer, breast cancer, gastric cancer, colorectal cancer, renal cancer, bladder cancer, ovarian cancer, prostate cancer, leukemia, lymphoma, and multiple myeloma.

In some embodiments, there is provided a method of treating lung cancer in an individual comprising administering to the individual: a) an effective amount of a composition comprising nanoparticles comprising a taxane (such as paclitaxel) and an albumin, and b) an effective amount of ABT-263. In some embodiments, there is provided a method of treating lung cancer in an individual comprising administering to the individual: a) an effective amount of a composition comprising nanoparticles comprising paclitaxel coated with an albumin (such as nab-paclitaxel for example Abraxane), and b) an effective amount of ABT-263. In some embodiments, the nanoparticle composition is administered intravenously. In some embodiments, the ABT-263 is administered orally. In some embodiments, the method further comprises administering to the individual an effective amount of carboplatin.

In some embodiments, the lung cancer is a non-small cell lung cancer (NSCLC). Examples of NCSLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC may be, according to TNM classifications, a stage T tumor (primary tumor), a stage N tumor (regional lymph nodes), or a stage M tumor (distant metastasis).

In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the lung cancer is small cell lung cancer (SCLC; also called oat cell carcinoma). The small cell lung cancer may be limited-stage, extensive stage or recurrent small cell lung cancer. In some embodiments, the individual may be a human who has a gene, genetic mutation, or polymorphism suspected or shown to be associated with lung cancer (e.g., SASH1, LATS1, IGF2R, PARK2, KRAS, PTEN, Kras2, Krag, Pasl, ERCC1, XPD, IL8RA, EGFR, α₁-AD, EPHX, MMP1, MMP2, MMP3, MMP12, IL1β, RAS, and/or AKT) or has one or more extra copies of a gene associated with lung cancer.

In some embodiments, there is provided a method of treating lung cancer (such as NSCLC, for example squamous NSCLC or adenocarcinoma NSCLC) in an individual, comprising administering to the individual: a) an effective amount of a composition comprising nanoparticles comprising a taxane (such as paclitaxel) and an albumin, wherein the taxane is in the dosage range of about 60 to about 300 mg/m² (including for example about 80 to about 200 mg/m², for example about 80 to about 150 mg/m² or about 80 to about 125 mg/m²), and b) an effective amount of ABT-263, wherein the ABT-263 is in the dosage range of about 10 to about 300 mg/day (including about 50 to about 200 mg/day, for example about 100 to about 200 mg/day). In some embodiments, there is provided a method of treating lung cancer (such as NSCLC, for example squamous NSCLC or adenocarcinoma NSCLC) in an individual, comprising administering to the individual: a) an effective amount of a composition comprising nanoparticles comprising paclitaxel coated with an albumin (such as nab-paclitaxel for example Abraxane®) and an albumin, wherein the paclitaxel coated with an albumin is in the dosage range of about 60 to about 300 mg/m² (including for example about 80 to about 200 mg/m², for example about 80 to about 150 mg/m² or about 80 to about 125 mg/m²), and b) an effective amount of ABT-263, wherein the ABT-263 is in the dosage range of about 10 to about 300 mg/day (including about 50 to about 200 mg/day, for example about 100 to about 200 mg/day). In some embodiments, there is provided a method of treating lung cancer (such as NSCLC, for example squamous NSCLC or adenocarcinoma NSCLC) in an individual, comprising administering to the individual: a) an effective amount of a composition comprising nanoparticles comprising paclitaxel coated with an albumin (such as nab-paclitaxel for example Abraxane®), wherein the taxane is in the dosage range of about 80 to about 125 mg/m² (such as any of about 80, 100, or 125 mg/m²) and b) about 100 to about 200 mg/day ABT-263. In some embodiments, the nanoparticle composition is administered first followed by administration of the ABT-263. In some embodiments, the ABT-263 is administered first followed by administration of the nanoparticle composition. In some embodiments, the administration of ABT-263 is synchronized with at least one dose of nanoparticle composition. In some embodiments, the administration of ABT-263 is synchronized with every dose of nanoparticle composition. In some embodiments, the nanoparticle composition is administered on a two, three, or four week dosing cycle, and the ABT-263 is administered 1, 2, 3, or 4 days out of the first four days of the dosing cycle. In some embodiments, the nanoparticle composition is administered two out of three weeks on a three week cycle (for example on days 1 and 8 of a three week cycle). In some embodiments, the nanoparticle composition is administered three out of four weeks on a four week cycle (for example on days 1, 8, and 15 of a four week cycle). In some embodiments the nanoparticle composition is administered two out of four weeks on a four week cycle (for example on days 1 and 15 of a four week cycle). In some embodiments, the nanoparticle composition is administered weekly on a four week cycle (for example on days 1, 8, 15, and 21 of a four-week cycle). In some embodiments, the nanoparticle composition is administered weekly on a three week cycle (for example on days 1, 8, and 15 of a three-week cycle). In some embodiments, the method further comprises administering to the individual an effective amount of carboplatin, for example on day 1 of the dosing cycle at the dosage of AUC=2 to AUC=6 (such as AUC=6).

In some embodiments, there is provided a method of treating pancreatic cancer in an individual comprising administering to the individual: a) an effective amount of a composition comprising nanoparticles comprising a taxane (such as paclitaxel) and an albumin, and b) an effective amount of ABT-263. In some embodiments, there is provided a method of treating pancreatic cancer in an individual comprising administering to the individual: a) an effective amount of a composition comprising nanoparticles comprising paclitaxel coated with an albumin (such as nab-paclitaxel for example Abraxane), and b) an effective amount of ABT-263. In some embodiments, the nanoparticle composition is administered intravenously. In some embodiments, the ABT-263 is administered orally. In some embodiments, the method further comprises administration of an effective amount of gemcitabine.

Pancreatic cancer that can be treated includes, but is not limited to, exocrine pancreatic cancer or endocrine pancreatic cancer. The exocrine pancreatic cancer includes, but is not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. The endocrine pancreatic cancer includes, but is not limited to, insulinomas and glucagonomas.

In some embodiments, the pancreatic cancer is early stage pancreatic cancer, non-metastatic pancreatic cancer, primary pancreatic cancer, advanced pancreatic cancer, locally advanced pancreatic cancer, metastatic pancreatic cancer, unresectable pancreatic cancer, pancreatic cancer in remission, or recurrent pancreatic cancer. In some embodiments, the pancreatic cancer is locally advanced pancreatic cancer, unresectable pancreatic cancer, or metastatic pancreatic ductal carcinoma. In some embodiments, the pancreatic cancer is, according to American Joint Committee on Cancer (AJCC) TNM classifications, a stage 0 tumor (the tumor is confined to the top layers of pancreatic duct cells and has not invaded deeper tissues, and it has not spread outside of the pancreas (e.g., pancreatic carcinoma in situ or pancreatic intraepithelial neoplasia III), a stage IA tumor (the tumor is confined to the pancreas and is less than 2 cm in size, and it has not spread to nearby lymph nodes or distinct sites), a stage IB tumor (the tumor is confined to the pancreas and is larger than 2 cm in size, and it has not spread to nearby lymph nodes or distant sites), a stage IIA tumor (the tumor is growing outside the pancreas but not into large blood vessels, and it has not spread to nearby lymph nodes or distant sites), stage IIB (the tumor is either confined to the pancreas or growing outside the pancreas but not into nearby large blood vessels or major nerves, and it has spread to nearby lymph nodes but not distant sites), stage III (the tumor is growing outside the pancreas into nearby large blood vessels or major nerves, and it may or may not have spread to nearby lymph nodes. It has not spread to distant sites) or stage IV tumor (the cancer has spread to distant sites).

In some embodiments, the individual is a human who exhibits one or more symptoms associated with pancreatic cancer. In some embodiments, the individual is at an early stage of pancreatic cancer. In some embodiments, the individual is at an advanced stage of pancreatic cancer. In some embodiments, the individual has non-metastatic pancreatic cancer. In some embodiments, the individual has primary pancreatic cancer. In some of embodiments, the individual is genetically or otherwise predisposed (e.g., having a risk factor) to developing pancreatic cancer. These risk factors include, but are not limited to, age, sex, race, diet, history of previous pancreatic cancer, presence of hereditary pancreatic cancer syndrome (e.g., BRCA2 mutation, familial atypical multiple mole melanoma, Peutz-Jeghers Syndrome, hereditary pancreatitis), genetic (e.g., familial pancreatic cancer) considerations, and environmental exposure. In some embodiments, the individuals at risk for pancreatic cancer include, e.g., those having at least 2 first-degree relatives who have experienced pancreatic cancer without accumulation of other cancers or familial diseases, and those whose risk is determined by analysis of genetic or biochemical markers (e.g., BRCA2, p16, STK11/LKB1, or PRSS1 gene). In some embodiments, the individual is positive for SPARC expression (for example based on IHC standard). In some embodiments, the individual is negative for SPARC expression.

Thus, for example, in some embodiments, there is provided a method of treating pancreatic cancer (such as metastatic or locally advanced pancreatic cancer) in an individual, comprising administering to the individual: a) an effective amount of a composition comprising nanoparticles comprising a taxane (such as paclitaxel) and an albumin, wherein the taxane is in the dosage range of about 60 to about 300 mg/m² (including for example about 80 to about 200 mg/m², for example about 80 to about 150 mg/m² or about 80 to about 125 mg/m²), and b) an effective amount of ABT-263, wherein the ABT-263 is in the dosage range of about 10 to about 300 mg/day (including about 50 to about 200 mg/day, for example about 100 to about 200 mg/day). In some embodiments, there is provided a method of treating pancreatic cancer (such as metastatic or locally advanced pancreatic cancer) in an individual, comprising administering to the individual: a) an effective amount of a composition comprising nanoparticles comprising paclitaxel coated with an albumin (such as nab-paclitaxel for example Abraxane®) and an albumin, wherein the paclitaxel coated with an albumin is in the dosage range of about 60 to about 300 mg/m² (including for example about 80 to about 200 mg/m², for example about 80 to about 150 mg/m² or about 80 to about 125 mg/m²), and b) an effective amount of ABT-263, wherein the ABT-263 is in the dosage range of about 10 to about 300 mg/day (including about 50 to about 200 mg/day, for example about 100 to about 200 mg/day). In some embodiments, there is provided a method of treating pancreatic cancer (such as metastatic or locally advanced pancreatic cancer) in an individual, comprising administering to the individual: a) an effective amount of a composition comprising nanoparticles comprising paclitaxel coated with an albumin (such as nab-paclitaxel for example Abraxane®), wherein the taxane is in the dosage range of about 80 to about 125 mg/m² (such as any of about 80, 100, or 125 mg/m²) and b) about 100 to about 200 mg/day ABT-263. In some embodiments, the pancreatic cancer is adenocarcinoma of the pancreas. In some embodiments, the nanoparticle composition is administered first followed by administration of the ABT-263. In some embodiments, the ABT-263 is administered first followed by administration of the nanoparticle composition. In some embodiments, the nanoparticle composition is administered on a two, three, or four week dosing cycle, and the ABT-263 is administered 1, 2, 3, or 4 days out of the first four days of the dosing cycle. In some embodiments, the nanoparticle composition is administered two out of three weeks on a three week cycle (for example on days 1 and 8 of a three week cycle). In some embodiments, the nanoparticle composition is administered three out of four weeks on a four week cycle (for example on days 1, 8, and 15 of a four week cycle). In some embodiments the nanoparticle composition is administered two out of four weeks on a four week cycle (for example on days 1 and 15 of a four week cycle). In some embodiments, the nanoparticle composition is administered weekly on a four week cycle (for example on days 1, 8, 15, and 21 of a four-week cycle). In some embodiments, the nanoparticle composition is administered weekly on a three week cycle (for example on days 1, 8, and 15 of a three-week cycle). In some embodiments, the method further comprises administering to the individual an effective amount of gemcitabine, for example administered immediately after the administration of the nanoparticle composition, such as at the dosage of about 1000 to about 2000 mg/m².

In some embodiments, there is provided a method of treating breast cancer (for example, HER2 negative breast cancer or for example, triple negative breast cancer) in an individual comprising administering to the individual: a) an effective amount of a composition comprising nanoparticles comprising a taxane (such as paclitaxel) and an albumin, and b) an effective amount of ABT-263. In some embodiments, there is provided a method of treating breast cancer (for example, HER2 negative breast cancer or for example, triple negative breast cancer) in an individual comprising administering to the individual: a) an effective amount of a composition comprising nanoparticles comprising paclitaxel coated with an albumin (such as nab-paclitaxel for example Abraxane), and b) an effective amount of ABT-263. In some embodiments, the nanoparticle composition is administered intravenously. In some embodiments, the ABT-263 is administered orally.

Breast cancer described herein can include early stage breast cancer, non-metastatic breast cancer, stage IV breast cancer, locally advanced breast cancer, metastatic breast cancer, hormone receptor positive metastatic breast cancer, breast cancer in remission, breast cancer in an adjuvant setting, ductal carcinoma in situ (DCIS), invasive ductal carcinoma (IDC), or breast cancer in a neoadjuvant setting. In some embodiments, the breast cancer is hormone receptor positive metastatic breast cancer. In some embodiments, the breast cancer (which may be HER2 positive or HER2 negative) is advanced breast cancer. In some embodiments, the breast cancer is ductal carcinoma in situ. In some embodiments, the individual may be a human who has a gene, genetic mutation, or polymorphism associated with breast cancer (e.g., BRCA1, BRCA2, ATM, CHEK2, RAD51, AR, DIRAS3, ERBB2, TP53, AKT, PTEN, and/or PI3K) or has one or more extra copies of a gene (e.g., one or more extra copies of the HER2 gene) associated with breast cancer.

The methods described herein can be used, for example, to treat, stabilize, prevent, and/or delay any type or stage of breast cancer, such as early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some embodiments, the method is useful for preoperative systemic therapy (PST).

In some embodiments, there is provided a method of treating breast cancer (which may be HER2 positive or HER2 negative), including, for example, advanced breast cancer, stage IV breast cancer, locally advanced breast cancer, and metastatic breast cancer. In some embodiments, the breast cancer is luminal type B breast cancer. In some embodiments, the breast cancer is basal cell breast cancer. In some embodiments, the individual is diagnosed with T2, T3, or T4 lesion, or a stage N, M0 or T1c, N1-3 and M0. In some embodiments, the individual has an ECOG performance status of 0-1. In some embodiments, the individual has skin metastasis to the ipsilateral breast. In some embodiments, the individual has undergone prior therapy (such as hormonal therapy). In some embodiments, the individual has not undergone prior therapy (such as hormonal therapy). In some embodiments, the individual is awaiting definitive surgery. In some embodiments, the breast cancer is resected breast cancer. In some embodiments, the breast cancer is unresected breast cancer, such as unresected stage II or III breast cancer.

In some embodiments, the method is for treating an individual having one or more of these risk factors resulting in a higher probability of developing breast cancer than an individual without these risk factor(s). These risk factors include, but are not limited to, age, sex, race, diet, history of previous disease, presence of precursor disease, genetic (i.e., hereditary) considerations, and environmental exposure. In some embodiments, the individual may be a human who is genetically or otherwise predisposed to developing breast cancer who has or has not been diagnosed with breast cancer. Individuals at risk for breast cancer include, e.g., those having relatives who have experienced this disease, and those whose risk is determined by analysis of genetic or biochemical markers. For example, the individual may be a human who has a gene, genetic mutation, or polymorphism associated with breast cancer (e.g., BRCA1, BRCA2, ATM, CHEK2, RAD51, AR, DIRAS3, ERBB2, and/or TP53) or has one or more extra copies of a gene (e.g., one or more extra copies of the HER2 gene) associated with breast cancer. In some embodiments, the breast cancer is HER2 negative. In some embodiments, the breast cancer is ER negative. In some embodiments, the breast cancer is PR negative. In some embodiments, the breast cancer is EP negative and HER2 negative. In some embodiments, the breast cancer is PR negative and HER2 negative. In some embodiments, the breast cancer is ER negative and PR negative. In some embodiment, the breast cancer is ER negative, PR negative, and HER2 negative.

In some embodiments, there is provided a method of treating breast cancer (for example, HER2 negative breast cancer or for example, triple negative breast cancer) in an individual, comprising administering to the individual: a) an effective amount of a composition comprising nanoparticles comprising a taxane (such as paclitaxel) and an albumin, wherein the taxane is in the dosage range of about 60 to about 300 mg/m² (including for example about 80 to about 200 mg/m², for example about 80 to about 150 mg/m² or about 80 to about 125 mg/m²), and b) an effective amount of ABT-263, wherein the ABT-263 is in the dosage range of about 10 to about 300 mg/day (including about 50 to about 200 mg/day, for example about 100 to about 200 mg/day). In some embodiments, there is provided a method of treating breast cancer (for example, HER2 negative breast cancer or for example, triple negative breast cancer) in an individual, comprising administering to the individual: a) an effective amount of a composition comprising nanoparticles comprising paclitaxel coated with an albumin (such as nab-paclitaxel for example Abraxane®) and an albumin, wherein the paclitaxel coated with an albumin is in the dosage range of about 60 to about 300 mg/m² (including for example about 80 to about 200 mg/m², for example about 80 to about 150 mg/m² or about 80 to about 125 mg/m2), and b) an effective amount of ABT-263, wherein the ABT-263 is in the dosage range of about 10 to about 300 mg/day (including about 50 to about 200 mg/day, for example about 100 to about 200 mg/day). In some embodiments, there is provided a method of treating breast cancer (for example, HER2 negative breast cancer or for example, triple negative breast cancer) in an individual, comprising administering to the individual: a) an effective amount of a composition comprising nanoparticles comprising paclitaxel coated with an albumin (such as nab-paclitaxel for example Abraxane®), wherein the taxane is in the dosage range of about 80 to about 125 mg/m² (such as any of about 80, 100, or 125 mg/m²) and b) about 100 to about 200 mg/day ABT-263. In some embodiments, the nanoparticle composition is administered first followed by administration of the ABT-263. In some embodiments, the ABT-263 is administered first followed by administration of the nanoparticle composition. In some embodiments, the nanoparticle composition is administered on a two, three, or four week dosing cycle, and the ABT-263 is administered 1, 2, 3, or 4 days out of the first four days of the dosing cycle. In some embodiments, the nanoparticle composition is administered two out of three weeks on a three week cycle (for example on days 1 and 8 of a three week cycle). In some embodiments, the nanoparticle composition is administered three out of four weeks on a four week cycle (for example on days 1, 8, and 15 of a four week cycle). In some embodiments the nanoparticle composition is administered two out of four weeks on a four week cycle (for example on days 1 and 15 of a four week cycle). In some embodiments, the nanoparticle composition is administered weekly on a four week cycle (for example on days 1, 8, 15, and 21 of a four-week cycle). In some embodiments, the nanoparticle composition is administered weekly on a three week cycle (for example on days 1, 8, and 15 of a three-week cycle).

In some embodiments of any of the methods described above, the taxane is paclitaxel. In some embodiments of any of the methods described above, the average diameter of the nanoparticles in the composition is no greater than about 200 nm. In some embodiments, the weight ratio of the albumin and the taxane in the nanoparticle composition is less than about 1:1 to about 18:1. In some embodiments of any of the methods described above, the individual is a human.

The combination therapy methods described herein are useful for treating cancers. The methods require administration of the nanoparticle composition and ABT-263 in effective amounts. In some embodiments, an effective amount is an amount sufficient to delay development. In some embodiments, an effective amount is an amount sufficient to prevent or delay recurrence. An effective amount can be administered in one or more administrations. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Thus, in some embodiments, there is provided a method of inhibiting cell proliferation (such as tumor growth) in an individual, comprising administering to the individual: a) an effective amount of a composition comprising nanoparticles comprising taxane and an albumin, and b) an effective amount of ABT-263. In some embodiments, the effective amounts of the taxane nanoparticle composition and the ABT-263 synergistically inhibit cell proliferation (such as tumor cell growth). In some embodiments, at least about 10% (including for example at least about any of 20%, 30%, 40%, 60%, 70%, 80%, 90%, or 100%) cell proliferation is inhibited. In some embodiments, the taxane is paclitaxel. In some embodiments, the taxane in the nanoparticle in the composition is administered by intravenous administration. In some embodiments, ABT-263 is administered by oral administration. In some embodiments, the ABT-263 is administered orally.

In some embodiments, there is provided a method of inhibiting tumor metastasis (such as metastasis of breast cancer, pulmonary metastasis or metastasis to the lymph node) in an individual, comprising administering to the individual: a) an effective amount of a composition comprising nanoparticles comprising taxane and an albumin, and b) an effective amount of ABT-263. In some embodiments, the effective amounts of the taxane nanoparticle composition and the ABT-263 synergistically inhibit tumor metastasis. In some embodiments, at least about 10% (including for example at least about any of 20%, 30%, 40%, 60%, 70%, 80%, 90%, or 100%) metastasis is inhibited. In some embodiments, method of inhibiting metastasis to lymph node is provided. In some embodiments, method of inhibiting metastasis to the lung is provided. In some embodiments, the taxane is paclitaxel. In some embodiments, the taxane in the nanoparticle in the composition is administered by intravenous administration. In some embodiments, the ABT-263 is administered orally.

In some embodiments, there is provided a method of reducing (such as eradiating) pre-existing tumor metastasis (such as pulmonary metastasis or metastasis to the lymph node) in an individual, comprising administering to the individual: a) an effective amount of a composition comprising nanoparticles comprising taxane and an albumin, and b) an effective amount of ABT-263. In some embodiments, the effective amounts of the taxane nanoparticle composition and the ABT-263 synergistically reduces (such as eradicates) tumor metastasis. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, method of reducing metastasis to the lung is provided. In some embodiments, the taxane is paclitaxel. In some embodiments, the taxane in the nanoparticle in the composition is administered by intravenous administration. In some embodiments, the ABT-263 is administered orally.

In some embodiments, there is provided a method of reducing incidence or burden of preexisting tumor metastasis (such as pulmonary metastasis or metastasis to the lymph node) in an individual, comprising administering to the individual: a) an effective amount of a composition comprising nanoparticles comprising taxane and an albumin, and b) an effective amount of ABT-263. In some embodiments, the taxane is paclitaxel. In some embodiments, the taxane in the nanoparticle in the composition is administered by intravenous administration. In some embodiments, the ABT-263 is administered orally.

In some embodiments, there is provided a method of reducing tumor size in an individual, comprising administering to the individual: a) an effective amount of a composition comprising nanoparticles comprising taxane and an albumin, and b) an effective amount of ABT-263. In some embodiments, the effective amounts of the taxane nanoparticle composition and the ABT-263 synergistically reduces tumor size. In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the taxane in the nanoparticle in the composition is administered by intravenous administration. In some embodiments, the ABT-263 is administered orally.

In some embodiments, there is provided a method of prolonging time to disease progression of cancer in an individual, comprising administering to the individual: a) an effective amount of a composition comprising nanoparticles comprising taxane and an albumin, and b) an effective amount of ABT-263. In some embodiments, the method prolongs the time to disease progression by at least any of 1, 2, 3, 4, 5, 6, 7, 8, 9, 10, 11, or 12 weeks. In some embodiments, the taxane is paclitaxel. In some embodiments, the taxane in the nanoparticle in the composition is administered by intravenous administration. In some embodiments, the ABT-263 is administered orally.

In some embodiments, there is provided a method of prolonging survival of an individual having cancer, comprising administering to the individual: a) an effective amount of a composition comprising nanoparticles comprising taxane and an albumin, and b) an effective amount of ABT-263. In some embodiments, the method prolongs the survival of the individual by at least any of 1, 2, 3, 4, 5, 6, 7, 8, 9, 10, 11, 12, 18, or 24 month. In some embodiments, the taxane is paclitaxel. In some embodiments, the taxane in the nanoparticle in the composition is administered by intravenous administration. In some embodiments, the ABT-263 is administered orally.

In some embodiments, the method is used to treat a primary tumor. In some embodiments, a method of treating metastatic cancer (that is, cancer that has metastasized from the primary tumor) is provided. In some embodiments, the method is for the treatment of an advanced disease or a lesser extent of disease, such as low tumor burden. In some embodiments, there is provided a method of treating cancer at an advanced stage. In some embodiments, the method is for the treatment of an early stage breast cancer. The methods may be practiced in an adjuvant setting. The methods provided herein may also be practiced in a neoadjuvant setting, i.e., the method may be carried out before the primary/definitive therapy. In some embodiments, the method further comprises conducting surgery on the individual following the completion of the treatment. For example, in some embodiments when the cancer is breast cancer, breast conserving surgery or mastectomy can be carried out within about 2, 3, 4, 5, 6, 7, 8, 9, 10, 11, or 12 weeks after completion of the neoadjuvant chemotherapy.

In some embodiments, the individual has previously been treated. In some embodiments, the individual has not previously been treated. In some embodiments, the treatment is a first line therapy. In some embodiments, the cancer has reoccurred after a remission.

The present application also provides pharmaceutical compositions comprising nanoparticles comprising a taxane (such as paclitaxel) and an albumin for use in the treatment of cancer, wherein said use comprises simultaneous, sequential, and/or concurrent administration of ABT-263. In some embodiments, the invention provides a pharmaceutical composition comprising ABT-263 for use in the treatment of cancer, wherein said use comprises simultaneous, sequential, and/or concurrent administration of a composition comprising nanoparticles comprising a taxane (such as paclitaxel) and an albumin.

In some embodiments, there is provided a kit comprising: a) a composition comprising nanoparticles comprising a taxane (such as paclitaxel) and an albumin, and b) an effective amount of ABT-263. In some embodiments, there is provided a medicine comprising: a) a composition comprising nanoparticles comprising a taxane (such as paclitaxel) and an albumin, and b) an effective amount of ABT-263.

In some embodiments, the levels of Bcl-xL, Bcl-2, and/or Mcl-1 can be used as a basis for selecting patients for treatment. The levels of Bcl-xL, Bcl-2, and/or Mcl-1 can be used, for example, for determining (and aiding assessment) in any one or more of the following: a) probable or likely suitability of an individual to initially receive treatment; b) probable or likely unsuitability of an individual to initially receive treatment(s); c) responsiveness to treatment; d) probable or likely suitability of an individual to continue to receive treatment; e) probable or likely unsuitability of an individual to receive treatment(s); 0 adjusting dosages; g) predicting likelihood of clinical benefits. The present application encompasses any of these methods.

For example, in some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-xL. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the level of Bcl-xL is used as a basis for selecting the individual for treatment. In some embodiments, the individual is selected for treatment if the individual has a high level of Bcl-xL. In some embodiments, the individual is selected for treatment if the individual has a low level of Bcl-xL. In some embodiments, the level of Bcl-xL is determined by immunohistochemistry method. In some embodiments, the level of the Bcl-xL is based on protein expression level. In some embodiments, the level of the Bcl-xL is based on mRNA level. In some embodiments, the method further comprises determining the level of the Bcl-xL prior to the treatment. In some embodiments, the method further comprises selecting the individual for treatment based on the Bcl-xL level.

In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-2. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the level of Bcl-2 is used as a basis for selecting the individual for treatment. In some embodiments, the individual is selected for treatment if the individual has a high level of Bcl-2. In some embodiments, the individual is selected for treatment if the individual has a low level of Bcl-2. In some embodiments, the level of Bcl-2 is determined by immunohistochemistry method. In some embodiments, the level of the Bcl-2 is based on protein expression level. In some embodiments, the level of the Bcl-2 is based on mRNA level. In some embodiments, the method further comprises determining the level of the Bcl-2 prior to the treatment. In some embodiments, the method further comprises selecting the individual for treatment based on the Bcl-2 level.

In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a low level of Mcl-1. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the level of Bcl-2 is used as a basis for selecting the individual for treatment. In some embodiments, the individual is selected for treatment if the individual has a low level of Mcl-1. In some embodiments, the level of Mcl-1 is determined by immunohistochemistry method. In some embodiments, the level of the Mcl-1 is based on protein expression level. In some embodiments, the level of the Mcl-1 is based on mRNA level. In some embodiments, the method further comprises determining the level of the Mcl-1 prior to the treatment. In some embodiments, the method further comprises selecting the individual for treatment based on the Mcl-1 level.

In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-xL and a low level of Bcl-2. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-xL and a high level of Bcl-2. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the levels of Bcl-xL and Bcl-2 are used as bases for selecting the individual for treatment. In some embodiments, the individual is selected for treatment if the individual has a high level of Bcl-xL and a low level of Bcl-2. In some embodiments, the individual is selected for treatment if the individual has a low level of Bcl-xL and a high level of Bcl-2. In some embodiments, the individual is selected for treatment if the individual has a high level of Bcl-xL and a high level of Bcl-2. In some embodiments, the levels of Bcl-xL and Bcl-2 are determined by immunohistochemistry method. In some embodiments, the levels of Bcl-xL and Bcl-2 are based on protein expression level. In some embodiments, the levels of Bcl-xL and Bcl-2 are based on mRNA level. In some embodiments, the method further comprises determining the levels of the Bcl-xL or Bcl-2 prior to the treatment. In some embodiments, the method further comprises selecting the individual for treatment based on the Bcl-xL and Bcl-2 levels.

In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-xL and a low level of Mcl-1. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the levels of Bcl-xL and Mcl-1 are used as bases for selecting the individual for treatment. In some embodiments, the individual is selected for treatment if the individual has a high level of Bcl-xL and a low level of Mcl-1. In some embodiments, the levels of Bcl-xL and Mcl-1 are determined by immunohistochemistry method. In some embodiments, the levels of Bcl-xL and Mcl-1 are based on protein expression level. In some embodiments, the levels of Bcl-xL and Mcl-1 are based on mRNA level. In some embodiments, the method further comprises determining the levels of the Bcl-xL or Mcl-1 prior to the treatment. In some embodiments, the method further comprises selecting the individual for treatment based on the Bcl-xL and Mcl-1 levels.

In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-xL, a high level of Bcl-2, and a low level of Mcl-1. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the levels of Bcl-xL, Bcl-2, and Mcl-1 are used as bases for selecting the individual for treatment. In some embodiments, the individual is selected for treatment if the individual has a high level of Bcl-xL, a high level of Bcl-2, and a low level of Mcl-1. In some embodiments, the levels of Bcl-xL, Bcl-2 and Mcl-1 are determined by immunohistochemistry method. In some embodiments, the levels of Bch xL, Bcl-2, and Mcl-1 are based on protein expression level. In some embodiments, the levels of Bcl-xL, Bcl-2, and Mcl-1 are based on mRNA level. In some embodiments, the method further comprises determining the levels of the Bcl-xL, Bcl-2, or Mcl-1 prior to the treatment. In some embodiments, the method further comprises selecting the individual for treatment based on the Bcl-xL, Bcl-2, and Mcl-1 levels.

In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the individual has a high level of Bcl-xL, a high level of Bcl-2, a low level of Mcl-1, and wherein the individual has a high level of caspase-3 and/or a high level of caspase-8. In some embodiments, there is provided a method of treating cancer in an individual (such as a human individual) comprising a) administering (such as intravenously administering) to the individual an effective amount of a composition comprising nanoparticles comprising a taxane (such as paclitaxel) and an albumin (such as human albumin or human serum albumin), and b) administering (such as orally administering) to the individual an effective amount of ABT-263, wherein the levels of Bcl-xL, Bcl-2, and Mcl-1 and the levels of caspase-3 and/or caspase-8 are used as bases for selecting the individual for treatment. In some embodiments, the individual is selected for treatment if the individual has a high level of Bcl-xL, a high level of Bcl-2, and a low level of Mcl-1 and wherein the individual has a high level of caspase-3 and/or a high level of caspase-8. In some embodiments, the gene encoding caspase-3 is mutated and/or the gene encoding caspase-8 is mutated. In some embodiments, the gene encoding caspase-3 is overexpressed and/or the gene encoding caspase-8 is overexpressed. In some embodiments, the gene encoding caspase-3 is epigenetically expressed when it should be silenced and/or the gene encoding caspase-8 is epigenetically expressed when it should be silenced. In some embodiments, the individual is not selected for treatment if the individual has a high level of Bcl-xL, a high level of Bcl-2, and a low level of Mcl-1 and wherein the individual has a low level or absence caspase-3 and/or a low level or absence of caspase-8. In some embodiments, the gene encoding caspase-3 is mutated and/or the gene encoding caspase-8 is mutated. In some embodiments, the gene encoding caspase-3 is deleted and/or the gene encoding caspase-8 is deleted. In some embodiments, the gene encoding caspase-3 is epigenetically silenced and/or the gene encoding caspase-8 is epigenetically silenced. In some embodiments, the levels of Bcl-xL, Bcl-2 and Mcl-1 and the levels of caspase-3 and/or caspase-8 are determined by immunohistochemistry method. In some embodiments, the levels of Bcl-xL, Bcl-2, and Mcl-1 and the levels of caspase-3 and/or caspase-8 are based on protein expression level. In some embodiments, the levels of Bcl-xL, Bcl-2, and Mcl-1 and the levels of caspase-3 and/or caspase-8 are based on mRNA level. In some embodiments, the method further comprises determining the levels of the Bcl-xL, Bcl-2, or Mcl-1 and the levels of caspase-3 and/or caspase-8 prior to the treatment. In some embodiments, the method further comprises selecting the individual for treatment based on the Bcl-xL, Bcl-2, and Mcl-1 levels and caspase-3 and/or caspase-8 levels.

The levels of Bcl-xL, Bcl-2, Mcl-1, caspase-3, or caspase-8 may be a high level or a low level as compared to a control sample. In some embodiments, the level of the Bcl-xL, Bcl-2, Mcl-1, caspase-3, or caspase-8 in an individual is compared to the level of the Bcl-xL, Bcl-2, Mcl-1, caspase-3, or caspase-8 in a control sample. In some embodiments the level of the Bcl-xL, Bcl-2, Mcl-1, caspase-3, or caspase-8 in a subject is compared to the level of the Bcl-xL, Bcl-2, Mcl-1, caspase-3, or caspase-8 in multiple control samples. In some embodiments, multiple control samples are used to generate a statistic that is used to classify the level of the Bcl-xL, Bcl-2, Mcl-1, caspase-3, or caspase-8 in an individual with cancer.

In some embodiments, the DNA copy number is determined, and a high DNA copy number for the gene encoding the Bcl-xL, Bcl-2, Mcl-1, caspase-3, or caspase-8 (for example a high DNA copy number as compared to a control sample) is indicative of a high level of the Bcl-xL, Bcl-2, Mcl-1, caspase-3, or caspase-8.

The classification or ranking of the Bcl-xL, Bcl-2, Mcl-1, caspase-3, or caspase-8 level (i.e., high or low) may be determined relative to a statistical distribution of control levels. In some embodiments, the classification or ranking is relative to a control sample obtained from the individual. In some embodiment the levels of the Bcl-xL, Bcl-2, Mcl-1, caspase-3, or caspase-8 is classified or ranked relative to a statistical distribution of control levels. In some embodiments, the level of the Bcl-xL, Bcl-2, Mcl-1, caspase-3, or caspase-8 is classified or ranked relative to the level from a control sample obtained from the subject.

Control samples can be obtained using the same sources and methods as non-control samples. In some embodiments, the control sample is obtained from a different individual (for example an individual not having cancer and/or an individual sharing similar ethnic, age, and gender identity). In some embodiments when the sample is a tumor tissue sample, the control sample may be a non-cancerous sample from the same individual. In some embodiments, multiple control samples (for example from different individuals) are used to determine a range of levels of Bcl-xL, Bcl-2, Mcl-1, caspase-3, or caspase-8 in a particular tissue, organ, or cell population. In some embodiments, the control sample is a cultured tissue or cell that has been determined to be a proper control. In some embodiments, the control is a cell that does not express the Bcl-xL, Bcl-2, Mcl-1, caspase-3, or caspase-8. In some embodiments, a clinically accepted normal level in a standardized test is used as a control level for determining the Bcl-xL, Bcl-2, Mcl-1, caspase-3, or caspase-8 level. In some embodiments, the reference level of Bcl-xL, Bcl-2, Mcl-1, caspase-3, or caspase-8 in the subject is classified as high, medium or low according to a scoring system, such as an immunohistochemistry-based scoring system, for example an H-Score as further discussed herein. In some embodiments, the reference level of Bcl-xL, Bcl-2, Mcl-1, caspase-3, or caspase-8 in the subject is classified as a low sample when the H-Score is less than or equal to the overall median H-Score.

In some embodiments, the Bcl-xL, Bcl-2, Mcl-1, caspase-3, or caspase-8 level is determined by measuring the level of a Bcl-xL, Bcl-2, Mcl-1, caspase-3, or caspase-8 in an individual and comparing to a control or reference (e.g., the median level for the given patient population or level of a second individual). For example, if the level of Bcl-xL, Bcl-2, Mcl-1, caspase-3, or caspase-8 for the single individual is determined to be above the median level of the patient population, that individual is determined to have high expression of the Bcl-xL, Bcl-2, Mcl-1, caspase-3, or caspase-8. Alternatively, if the level of a Bcl-xL, Bcl-2, Mcl-1, caspase-3, or caspase-8 for the single individual is determined to be below the median level of the patient population, that individual is determined to have low expression of the Bcl-xL, Bcl-2, Mcl-1, caspase-3, or caspase-8. In some embodiments, the individual is compared to a second individual and/or a patient population which is responsive to treatment. In some embodiments, the individual is compared to a second individual and/or a patient population which is not responsive to treatment. In any of the embodiments herein, the levels are determined by measuring the level of Bcl-xL, Bcl-2, Mcl-1, caspase-3, or caspase-8. For example, if the level of an mRNA encoding Bcl-xL, Bcl-2, Mcl-1, caspase-3, or caspase-8 for the single individual is determined to be above the median level of the patient population, that individual is determined to have a high level of an mRNA encoding Bcl-xL, Bcl-2, Mcl-1, caspase-3, or caspase-8. Alternatively, if the level of mRNA encoding the Bcl-xL, Bcl-2, or Mcl-1 for the single individual is determined to be below the median level of the patient population, that individual is determined to have a low level of an mRNA encoding Bch xL, Bcl-2, Mcl-1, caspase-3, or caspase-8.

In some embodiments, the reference level of Bcl-xL, Bcl-2, Mcl-1, caspase-3, or caspase-8 is determined by obtaining a statistical distribution of Bcl-xL, Bcl-2, Mcl-1, caspase-3, or caspase-8 levels.

In some embodiments, bioinformatics methods are used for the determination and classification of the levels of Bcl-xL, Bcl-2, Mcl-1, caspase-3, or caspase-8. Numerous alternative bioinformatics approaches have been developed to assess gene set expression profiles using gene expression profiling data. Methods include but are not limited to those described in Segal, E. et al. Nat. Genet. 34:66-176 (2003); Segal, E. et al. Nat. Genet. 36:1090-1098 (2004); Barry, W. T. et al. Bioinformatics 21:1943-1949 (2005); Tian, L. et al. Proc Nat'l Acad Sci USA 102:13544-13549 (2005); Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, mRNA level is determined, and a low level is an mRNA level less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the level obtained from a control. In some embodiments, high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times to that of what is considered as clinically normal or to the level obtained from a control.

In some embodiments, protein expression level is determined, for example by immunohistochemistry. For example, the criteria for low or high levels can be made based on the number of positive staining cells and/or the intensity of the staining, for example by using an antibody that specifically recognizes the Bcl-xL, Bcl-2, Mcl-1, caspase-3, or caspase-8 protein. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining.

In some embodiments, the level is high if more than about 40%, 45%, 50%, 55%, 60%, 65%, 70%, 75%, 80%, 85%, or 90%, cells have positive staining. In some embodiments, the level is high if the staining is as intense as positive control staining. In some embodiments, the level is high if the staining is 80%, 85%, or 90% as intense as positive control staining.

In some embodiments, the scoring is based on an “H-score” as described in US Pat. Pub. No. 2013/0005678. An H-score is obtained by the formula: 3×percentage of strongly staining cells+2×percentage of moderately staining cells+percentage of weakly staining cells, giving a range of 0 to 300.

In some embodiments, strong staining, moderate staining, and weak staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

### Modes of Administration

The composition comprising nanoparticles comprising taxane (also referred to as “nanoparticle composition”) and ABT-263 can be administered simultaneously (i.e., simultaneous administration) and/or sequentially (i.e., sequential administration).

In some embodiments, the nanoparticle composition and the ABT-263 are administered simultaneously. The term “simultaneous administration,” as used herein, means that the nanoparticle composition and the ABT-263 are administered with a time separation of no more than about 15 minute(s), such as no more than about any of 10, 5, or 1 minutes. When the drugs are administered simultaneously, the drug in the nanoparticles and the ABT-263 may be contained in the same composition (e.g., a composition comprising both the nanoparticles and the ABT-263) or in separate compositions (e.g., the nanoparticles are contained in one composition and the ABT-263 is contained in another composition).

In some embodiments, the nanoparticle composition and the ABT-263 are administered sequentially. The term “sequential administration” as used herein means that the drug in the nanoparticle composition and the ABT-263 are administered with a time separation of more than about 15 minutes, such as more than about any of 20, 30, 40, 50, 60 or more minutes. Either the nanoparticle composition or the ABT-263 may be administered first. The nanoparticle composition and the ABT-263 are contained in separate compositions, which may be contained in the same or different packages.

In some embodiments, the administration of the nanoparticle composition and the ABT-263 are concurrent, i.e., the administration period of the nanoparticle composition and that of the ABT-263 overlap with each other. In some embodiments, the nanoparticle composition is administered for at least one cycle (for example, at least any of 2, 3, or 4 cycles) prior to the administration of the ABT-263. In some embodiments, the ABT-263 is administered for at least any of one, two, three, or four weeks. In some embodiments, the administrations of the nanoparticle composition and the ABT-263 are initiated at about the same time (for example, within any one of 1, 2, 3, 4, 5, 6, or 7 days). In some embodiments, the administrations of the nanoparticle composition and the ABT-263 are terminated at about the same time (for example, within any one of 1, 2, 3, 4, 5, 6, or 7 days). In some embodiments, the administration of the ABT-263 continues (for example for about any one of 1, 2, 3, 4, 5, 6, 7, 8, 9, 10, 11, or 12 months) after the termination of the administration of the nanoparticle composition. In some embodiments, the administration of the ABT-263 continues (for example for about any one of 1, 2, 3, 4, 5, 6, 7, 8, 9, 10, 11, 12, 13, or 14 days) after the termination of the administration of the nanoparticle composition. In some embodiments, the administration of the ABT-263 is initiated after (for example after about any one of 1, 2, 3, 4, 5, 6, 7, 8, 9, 10, 11, or 12 months) the initiation of the administration of the nanoparticle composition. In some embodiments, the administration of the ABT-263 is initiated after (for example after about any one of 1, 2, 3, 4, 5, 6, 7, 8, 9, 10, 11, 12, 13, or 14 days) the initiation of the administration of the nanoparticle composition. In some embodiments, the administrations of the nanoparticle composition and the ABT-263 are initiated and terminated at about the same time. In some embodiments, the administrations of the nanoparticle composition and the ABT-263 are initiated at about the same time and the administration of the ABT-263 continues (for example for about any one of 1, 2, 3, 4, 5, 6, 7, 8, 9, 10, 11, or 12 months) after the termination of the administration of the nanoparticle composition. In some embodiments, the administrations of the nanoparticle composition and the ABT-263 are initiated at about the same time and the administration of the ABT-263 continues (for example for about any one of 1, 2, 3, 4, 5, 6, 7, 8, 9, 10, 11, 12, 13, or 14 days) after the termination of the administration of the nanoparticle composition. In some embodiments, the administration of the nanoparticle composition and the ABT-263 stop at about the same time and the administration of the ABT-263 is initiated after (for example after about any one of 1, 2, 3, 4, 5, 6, 7, 8, 9, 10, 11, or 12 months) the initiation of the administration of the nanoparticle composition. In some embodiments, the administration of the nanoparticle composition and the ABT-263 stop at about the same time and the administration of the ABT-263 is initiated after (for example after about any one of 1, 2, 3, 4, 5, 6, 7, 8, 9, 10, 11, 12, 13, or 14 days) the initiation of the administration of the nanoparticle composition.

In some embodiments, the administration of ABT-263 is synchronized with at least one dose of nanoparticle composition in a dosing cycle. “Synchronized” as described herein means that the first administration of ABT-263 in a dosing cycle is within 1, 2, or 3 days, or is on the same day as one of the administration of the nanoparticle composition. For example, in some embodiments, the nanoparticle composition is administered weekly on a three week cycle or a three out of four week cycle, and ABT-263 is administered on day 1; day 2; day 3; day 4; days 1 and 2; days 1 and 3; days 1 and 4; days 2 and 3; days 2 and 4; days 3 and 4; days 1, 2, and 3; days 1, 2, and 4; days 1, 3, and 4, days 2, 3, and 4; or days 1, 2, 3, and 4, of the first week of the dosing cycle. In some embodiments, the nanoparticle composition is administered weekly on a three week cycle or a three out of four week cycle, and ABT-263 is administered on day 1; day 2; day 3; day 4; days 1 and 2; days 1 and 3; days 1 and 4; days 2 and 3; days 2 and 4; days 3 and 4; days 1, 2, and 3; days 1, 2, and 4; days 1, 3, and 4, days 2, 3, and 4; or days 1, 2, 3, and 4, of the second week of the dosing cycle. In some embodiments, the nanoparticle composition is administered weekly on a three week cycle or a three out of four week cycle, and ABT-263 is administered on day 1; day 2; day 3; day 4; days 1 and 2; days 1 and 3; days 1 and 4; days 2 and 3; days 2 and 4; days 3 and 4; days 1, 2, and 3; days 1, 2, and 4; days 1, 3, and 4, days 2, 3, and 4; or days 1, 2, 3, and 4, of the third week of the dosing cycle.

The dosing frequency of the drug-containing nanoparticle composition and the ABT-263 may be adjusted over the course of the treatment, based on the judgment of the administering physician. When administered separately, the drug-containing nanoparticle composition and the ABT-263 can be administered at different dosing frequency or intervals. For example, the drug-containing nanoparticle composition can be administered weekly, while ABT-263 can be administered more or less frequently. In some embodiments, sustained continuous release formulation of the drug-containing nanoparticle and/or ABT-263 may be used. Various formulations and devices for achieving sustained release are known in the art. Exemplary dosing frequencies are further provided herein. The nanoparticle composition and the ABT-263 can be administered using the same route of administration or different routes of administration. Exemplary administration routes are further provided herein. In some embodiments (for both simultaneous and sequential administrations), the taxane in the nanoparticle composition and the ABT-263 are administered at a predetermined ratio. For example, in some embodiments, the ratio by weight of the taxane in the nanoparticle composition and the ABT-263 is about 1 to 1. In some embodiments, the weight ratio may be between about 0.001 to about 1 and about 1000 to about 1, or between about 0.01 to about 1 and 100 to about 1. In some embodiments, the ratio by weight of the taxane in the nanoparticle composition and the ABT-263 is less than about any of 100:1, 50:1, 30:1, 10:1, 9:1, 8:1, 7:1, 6:1, 5:1, 4:1, 3:1, 2:1, and 1:1 In some embodiments, the ratio by weight of the taxane in the nanoparticle composition and the ABT-263 is more than about any of 1:1, 2:1, 3:1, 4:1, 5:1, 6:1, 7:1, 8:1, 9:1, 30:1, 50:1, 100:1. Other ratios are contemplated.

The doses required for the taxane and/or the ABT-263 may (but not necessarily) be lower than what is normally required when each agent is administered alone. Thus, in some embodiments, a subtherapeutic amount of the drug in the nanoparticle composition and/or the ABT-263 is administered. “Subtherapeutic amount” or “subtherapeutic level” refer to an amount that is less than therapeutic amount, that is, less than the amount normally used when the drug in the nanoparticle composition and/or the ABT-263 are administered alone. The reduction may be reflected in terms of the amount administered at a given administration and/or the amount administered over a given period of time (reduced frequency).

In some embodiments, enough ABT-263 is administered so as to allow reduction of the normal dose of the drug in the nanoparticle composition required to effect the same degree of treatment by at least about any of 5%, 10%, 20%, 30%, 50%, 60%, 70%, 80%, 90%, or more. In some embodiments, enough drug in the nanoparticle composition is administered so as to allow reduction of the normal dose of the ABT-263 required to effect the same degree of treatment by at least about any of 5%, 10%, 20%, 30%, 50%, 60%, 70%, 80%, 90%, or more.

In some embodiments, the dose of both the taxane in the nanoparticle composition and the ABT-263 are reduced as compared to the corresponding normal dose of each when administered alone. In some embodiments, both the taxane in the nanoparticle composition and the ABT-263 are administered at a subtherapeutic, i.e., reduced, level. In some embodiments, the dose of the nanoparticle composition and/or the ABT-263 is substantially less than the established maximum toxic dose (MTD). For example, the dose of the nanoparticle composition and/or the ABT-263 is less than about 50%, 40%, 30%, 20%, or 10% of the MTD.

In some embodiments, the dose of taxane and/or the dose of the ABT-263 is higher than what is normally required when each agent is administered alone. For example, in some embodiments, the dose of the nanoparticle composition and/or the ABT-263 is substantially higher than the established maximum toxic dose (MTD). For example, the dose of the nanoparticle composition and/or the ABT-263 is more than about 50%, 40%, 30%, 20%, or 10% of the MTD of the agent when administered alone.

In some embodiments, the amount of a taxane (e.g., paclitaxel) in the composition is included in any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of a taxane (e.g., paclitaxel) or derivative thereof in the amount of the composition (e.g., a unit dosage form) is in the range of about 5 mg to about 500 mg, such as about 30 mg to about 300 mg or about 50 mg to about 200 mg. In some embodiments, the concentration of the taxane (e.g., paclitaxel) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane (e.g., paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary amounts of a taxane (e.g., paclitaxel) in the nanoparticle composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m² 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of a taxane (e.g., paclitaxel). In various embodiments, the composition includes less than about any of 350 mg/m², 300 mg/m², 250 mg/m², 200 mg/m² 150 mg/m², 120 mg/m², 100 mg/m², 90 mg/m², 50 mg/m², or 30 mg/m² of a taxane (e.g., paclitaxel). In some embodiments, the amount of the taxane (e.g., paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the amount of a taxane (e.g., paclitaxel) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the amount of a taxane (e.g., paclitaxel) in the composition is about 5 to about 300 mg/m², such as about 20 to about 300 mg/m², about 50 to about 250 mg/m², about 100 to about 150 mg/m², about 120 mg/m², about 130 mg/m², or about 140 mg/m², or about 260 mg/m².

In some embodiments of any of the above aspects, the amount of a taxane (e.g., paclitaxel) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, or 20 mg/kg. In various embodiments, the amount of a taxane (e.g., paclitaxel) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a taxane (e.g., paclitaxel).

Exemplary dosing frequencies for the nanoparticle composition (and as indicated below for the ABT-263) include, but are not limited to, weekly without break; weekly, three out of four weeks; once every three weeks; once every two weeks; weekly, two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week, or three times daily, two times daily. In some embodiments, the intervals between each administration are less than about any of 6 months, 3 months, 1 month, 20 days, 15 days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the taxane in the nanoparticle composition is administered weekly. In some embodiments, the taxane in a nanoparticle composition is administered every two weeks. In some embodiments, the taxane in the nanoparticle composition is administered every three weeks. In some embodiments, the ABT-263 is administered 1×, 2×, 3×, 4×, 5×, 6×, or 7 times a week. In some embodiments, the ABT-263 is administered every two weeks or two out of three weeks. In some embodiments, the taxane is paclitaxel.

The administration of the nanoparticle composition (and for the ABT-263) can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months. In some embodiments, the taxane (e.g., paclitaxel) is administered over a period of at least one month, wherein the interval between each administration is no more than about a week, and wherein the dose of the taxane (e.g., paclitaxel) at each administration is about 0.25 mg/m² to about 75 mg/m², such as about 0.25 mg/m² to about 25 mg/m² or about 25 mg/m² to about 50 mg/m².

In some embodiments, the dosage of a taxane (e.g., paclitaxel) in a nanoparticle composition can be in the range of 5-400 mg/m² when given on a 3 week schedule, or 5-250 mg/m² when given on a weekly schedule. For example, the amount of a taxane (e.g., paclitaxel) can be about 60 to about 300 mg/m² (e.g., about 260 mg/m²) when given on a three week schedule.

Other exemplary dosing schedules for the administration of the nanoparticle composition (e.g., paclitaxel/albumin nanoparticle composition) include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m², twice a week; and 150-250 mg/m² twice a week. The dosing frequency of the composition may be adjusted over the course of the treatment based on the judgment of the administering physician.

In some embodiments, the individual is treated for at least about any of one, two, three, four, five, six, seven, eight, nine, or ten treatment cycles. The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

Other exemplary dose of the taxane (in some embodiments paclitaxel) in the nanoparticle composition include, but is not limited to, about any of 50 mg/m², 60 mg/m², 75 mg/m², 80 mg/m² 90 mg/m², 100 mg/m² 120 mg/m² 160 mg/m², 175 mg/m², 200 mg/m², 210 mg/m², 220 mg/m², 260 mg/m², and 300 mg/m². For example, the dosage of paclitaxel in a nanoparticle composition can be in the range of about 100-400 mg/m² when given on a 3 week schedule, or about 50-250 mg/m² (such as about 80 to about 125 mg/m²) when given on a weekly schedule.

The dosing frequency of the ABT-263 can be the same or different from that of the nanoparticle composition. Exemplary frequencies are provided above. As further example, the ABT-263 can be administered three times a day, two times a day, daily, 6 times a week, 5 times a week, 4 times a week, 3 times a week, two times a week, weekly. In some embodiments, the ABT-263 is administered twice daily or three times daily. Exemplary amounts (for example daily amounts) of the ABT-263 include, but are not limited to, any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg, about 500 to about 550 mg, about 550 to about 600 mg, about 600 to about 650 mg, about 650 to about 700 mg, about 700 mg to about 800 mg, about 800 mg to about 850 mg, about 850 mg to about 900 mg, about 900 mg to about 950 mg, about 950 mg to about 1000 mg. For example, the ABT-263 can be administered at a dose of about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg).

Other suitable doses of ABT-263 include, for example, about 25 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 pm, the average dosage interval is 8 hours (a 24-hour time span divided by 3). If the drug is formulated as a discrete dosage form such as a tablet or capsule, a plurality (e.g., 2 to about 10) of dosage forms administered at one time is considered a unit dose for the purpose of defining the average dosage interval.

A daily dosage amount and dosage interval can, in some exemplary embodiments, be selected to maintain a plasma concentration of ABT-263 in a range of about 0.5 μg/ml to about 10 μg/ml. Thus, during a course of ABT-263 therapy according to such embodiments, the steady-state peak plasma concentration (Cmax) should in general not exceed about 10 μg/ml, and the steady-state trough plasma concentration (Cmin) should in general not fall below about 0.5 μg/ml. It will further be found desirable to select, within the ranges provided above, a daily dosage amount and average dosage interval effective to provide a Cmax/Cmin ratio not greater than about 5, for example not greater than about 3, at steady-state. It will be understood that longer dosage intervals will tend to result in greater Cmax/Cmin ratios. For example, at steady-state, an ABT-263 Cmax of about 3 to about 8 μg/ml and Cmin of about 1 μg/ml to about 5 μg/ml can be targeted. Steady-state values of Cmax and Cmin can be established in a human PK study, for example conducted according to standard protocols including but not limited to those acceptable to a regulatory agency such as the U.S. Food and Drug Administration (FDA).

In some embodiments, the amount of taxane in the nanoparticle composition is between about 45 mg/m² to about 350 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane in the nanoparticle composition is between about 80 mg/m² to about 350 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane in the nanoparticle composition is between about 80 mg/m² to about 300 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane in the nanoparticle composition is between about 150 mg/m² to about 350 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane in the nanoparticle composition is between about 80 mg/m² to about 150 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane (e.g., paclitaxel) in the nanoparticle composition is about 100 mg/m². In some embodiments, the amount of taxane in the nanoparticle composition is between about 170 mg/m² to about 200 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane in the nanoparticle composition is between about 200 mg/m² to about 350 mg/m² and the amount of the ABT-263 is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the amount of taxane (e.g., paclitaxel) in the nanoparticle composition is about 260 mg/m². In some embodiments of any of the above methods, the amount of the ABT-263 is about 20-30 mg/kg, about 30-40 mg/kg, about 40-50 mg/kg, about 50-60 mg/kg, about 60-70 mg/kg, about 70-80 mg/kg, about 80-100 mg/kg, or about 100-120 mg/kg.

In some embodiments, the amount of taxane in the nanoparticle composition is between about 45 mg/m² to about 350 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane in the nanoparticle composition is between about 80 mg/m² to about 350 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane in the nanoparticle composition is between about 80 mg/m² to about 300 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane in the nanoparticle composition is between about 150 mg/m² to about 350 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane in the nanoparticle composition is between about 80 mg/m² to about 150 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane in the nanoparticle composition is between about 170 mg/m² to about 200 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane in the nanoparticle composition is between about 200 mg/m² to about 350 mg/m² and the amount (for example daily amount) of the ABT-263 is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the amount of taxane (e.g., paclitaxel) in the nanoparticle composition is about 100 mg/m². In some embodiments of any of the above methods, the amount (for example daily amount) of the ABT-263 is about 100-200 mg, about 200-300 mg, about 300-400 mg, about 400-500 mg.

In some embodiments, the amount of paclitaxel in the nanoparticle composition is about 80 to about 200 mg/m² (such as about 80, about 90, about 100, about 110, about 120, about 130, about 140, about 150, about 160, about 170, about 180, about 190 or about 200 mg/m², including any range in between these values) and the amount of the ABT-263 is about 10 to about 350 mg (such as about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, or about 350 mg, including any range in between these values).

In some embodiments, the amount of paclitaxel in the nanoparticle composition is about 80-125 mg/m², (including for example about 90, about 100, about 110, or about 120 mg/m², including any range in between these values) and the amount of the ABT-263 is between 10-200 mg (including for example 10, 20, 25, 30, 40, 50, 60, 70, 75, 80, 90, 100, 110, 120, 125, 130, 140, 150, 160, 170, 175, 180, 190, 200 mg).

In some embodiments, the composition comprising nanoparticles comprising paclitaxel and albumin is administered once every three weeks in a 21 day cycle (q3w) and the ABT-263 is administered on days 1, 2, 3, and 4 at each cycle. In some embodiments, the composition comprising nanoparticles comprising paclitaxel and albumin is administered q3w and the ABT-263 is administered on days 1, 2, and 3. In some embodiments, the composition comprising nanoparticles comprising paclitaxel and albumin is administered q3w and the ABT-263 is administered on days 1 and 2. In some embodiments, the composition comprising nanoparticles comprising paclitaxel and albumin is administered q3w and the ABT-263 is administered on day 1. In some embodiments, the composition comprising nanoparticles comprising paclitaxel and albumin is administered q3w and the ABT-263 is administered on days 2, 3, and 4. In some embodiments, the composition comprising nanoparticles comprising paclitaxel and albumin is administered q3w and the ABT-263 is administered on days 1 and 3. In some embodiments, the composition comprising nanoparticles comprising paclitaxel and albumin is administered q3w and the ABT-263 is administered on days 2 and 4. In some embodiments, the composition comprising nanoparticles comprising paclitaxel and albumin is administered q3w and the ABT-263 is administered on day 2. In some embodiments, the composition comprising nanoparticles comprising paclitaxel and albumin is administered q3w and the ABT-263 is administered on days 3 and 4. In some embodiments, the composition comprising nanoparticles comprising paclitaxel and albumin is administered q3w and the ABT-263 is administered on days 2 and 3. In some embodiments, the composition comprising nanoparticles comprising paclitaxel and albumin is administered q3w and the ABT-263 is administered on day 4.

In some embodiments, the composition comprising nanoparticles comprising paclitaxel and albumin is administered two out of three weeks on days 1 and 8 (qwx2 (days 1 and 8) per 21 day cycle) and the ABT-263 is administered on days 1, 2, 3, and 4 of the 21 day cycle. In some embodiments, the composition comprising nanoparticles comprising paclitaxel and albumin is administered qwx2 (days 1 and 7) per 21 day cycle and the ABT-263 is administered on days 1, 2, and 3. In some embodiments, the composition comprising nanoparticles comprising paclitaxel and albumin is administered qwx2 (days 1 and 8) per 21 day cycle and the ABT-263 is administered on days 1 and 2. In some embodiments, the composition comprising nanoparticles comprising paclitaxel and albumin is administered qwx2 (days 1 and 8) per 21 day cycle and the ABT-263 is administered on day 1. In some embodiments, the composition comprising nanoparticles comprising paclitaxel and albumin is administered qwx2 (days 1 and 8) per 21 day cycle and the ABT-263 is administered on days 2, 3, and 4. In some embodiments, the composition comprising nanoparticles comprising paclitaxel and albumin is administered q3w and the ABT-263 is administered on days 1 and 3. In some embodiments, the composition comprising nanoparticles comprising paclitaxel and albumin is administered qwx2 (days 1 and 8) per 21 day cycle and the ABT-263 is administered on days 2 and 4. In some embodiments, the composition comprising nanoparticles comprising paclitaxel and albumin is administered qwx2 (days 1 and 8) per 21 day cycle and the ABT-263 is administered on day 2. In some embodiments, the composition comprising nanoparticles comprising paclitaxel and albumin is administered qwx2 (days 1 and 8) per 21 day cycle and the ABT-263 is administered on days 3 and 4. In some embodiments, the composition comprising nanoparticles comprising paclitaxel and albumin is administered qwx2 (days 1 and 8) per 21 day cycle and the ABT-263 is administered on days 2 and 3. In some embodiments, the composition comprising nanoparticles comprising paclitaxel and albumin is administered qwx2 (days 1 and 8) per 21 day cycle and the ABT-263 is administered on day 4.

In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8,9, 10, 11, 15, 16, 17, and 18 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8,9, 10, 11, 15, 16, and 17 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8,9, 10, 11, 15, and 16 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8,9, 10, 11, and 15 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8,9, 10, and 11 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8,9, and 10 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, 8, and 9 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, 4, and 8 of the 28 day cycle. In some embodiments, the composition comprising nanoparticles comprising paclitaxel and albumin is administered weekly, three out of four weeks on days 1, 8, and 15 (qwx3 (days 1, 8, and 15) per 28 day cycle) and the ABT-263 is administered on days 1, 2, 3, and 4 of the 28 day cycle. In some embodiments, the composition comprising nanoparticles comprising paclitaxel and albumin is administered qwx3 (days 1, 8, and 15) per 28 day cycle and the ABT-263 is administered on days 1, 2, and 3. In some embodiments, the composition comprising nanoparticles comprising paclitaxel and albumin is administered qwx3 (days 1, 8, and 15) per 28 day cycle and the ABT-263 is administered on days 1 and 2. In some embodiments, the composition comprising nanoparticles comprising paclitaxel and albumin is administered qwx3 (days 1, 8, and 15) per 28 day cycle and the ABT-263 is administered on day 1. In some embodiments, the composition comprising nanoparticles comprising paclitaxel and albumin is administered qwx3 (days 1, 8, and 15) per 28 day cycle and the ABT-263 is administered on days 2, 3, and 4. In some embodiments, the composition comprising nanoparticles comprising paclitaxel and albumin is administered q3w and the ABT-263 is administered on days 1 and 3. In some embodiments, the composition comprising nanoparticles comprising paclitaxel and albumin is administered qwx3 (days 1, 8, and 15) per 28 day cycle and the ABT-263 is administered on days 2 and 4. In some embodiments, the composition comprising nanoparticles comprising paclitaxel and albumin is administered qwx3 (days 1, 8, and 15) per 28 day cycle and the ABT-263 is administered on day 2. In some embodiments, the composition comprising nanoparticles comprising paclitaxel and albumin is administered qwx3 (days 1, 8, and 15) per 28 day cycle and the ABT-263 is administered on days 3 and 4. In some embodiments, the composition comprising nanoparticles comprising paclitaxel and albumin is administered qwx3 (days 1, 8, and 15) per 28 day cycle and the ABT-263 is administered on days 2 and 3. In some embodiments, the composition comprising nanoparticles comprising paclitaxel and albumin is administered qwx3 (days 1, 8, and 15) per 28 day cycle and the ABT-263 is administered on day 4.

In some embodiments, the composition comprising nanoparticles comprising paclitaxel and albumin is administered weekly, two out of four weeks on a four week cycle on days 1 and 15 (q2w (Days 1 and 15) per 28 day cycle) and the ABT-263 is administered on days 1, 2, 3, and 4. In some embodiments, the composition comprising nanoparticles comprising paclitaxel and albumin is administered q2w (Days 1 and 15) per 28 day cycle and the ABT-263 is administered on days 1, 2, and 3. In some embodiments, the composition comprising nanoparticles comprising paclitaxel and albumin is administered q2w (Days 1 and 15) per 28 day cycle and the ABT-263 is administered on days 1 and 2. In some embodiments, the composition comprising nanoparticles comprising paclitaxel and albumin is administered q2w (Days 1 and 15) per 28 day cycle and the ABT-263 is administered on day 1. In some embodiments, the composition comprising nanoparticles comprising paclitaxel and albumin is administered q2w (Days 1 and 15) per 28 day cycle and the ABT-263 is administered on days 2, 3, and 4. In some embodiments, the composition comprising nanoparticles comprising paclitaxel and albumin is administered q3w and the ABT-263 is administered on days 1 and 3. In some embodiments, the composition comprising nanoparticles comprising paclitaxel and albumin is administered q2w (Days 1 and 15) per 28 day cycle and the ABT-263 is administered on days 2 and 4. In some embodiments, the composition comprising nanoparticles comprising paclitaxel and albumin is administered q2w (Days 1 and 15) per 28 day cycle and the ABT-263 is administered on day 2. In some embodiments, the composition comprising nanoparticles comprising paclitaxel and albumin is administered q2w (Days 1 and 15) per 28 day cycle and the ABT-263 is administered on days 3 and 4. In some embodiments, the composition comprising nanoparticles comprising paclitaxel and albumin is administered q2w (Days 1 and 15) per 28 day cycle and the ABT-263 is administered on days 2 and 3. In some embodiments, the composition comprising nanoparticles comprising paclitaxel and albumin is administered q2w (Days 1 and 15) per 28 day cycle and the ABT-263 is administered on day 4.

In some embodiments, the composition comprising nanoparticles comprising paclitaxel and albumin is administered weekly, three out of four weeks on days 1, 8, and 15 of a four week cycle (qwx3 (days 1, 8, and 15) per 28 day cycle) and the ABT-263 is administered on days 1, 2, 3, and 4 of the 28 day cycle. In some embodiments, the composition comprising nanoparticles comprising paclitaxel and albumin is administered qwx3 (days 1, 8, and 15) per 28 day cycle and the ABT-263 is administered on days 1, 2, and 3. In some embodiments, the composition comprising nanoparticles comprising paclitaxel and albumin is administered qwx3 (days 1, 8, and 15) per 28 day cycle and the ABT-263 is administered on days 1 and 2. In some embodiments, the composition comprising nanoparticles comprising paclitaxel and albumin is administered qwx3 (days 1, 8, and 15) per 28 day cycle and the ABT-263 is administered on day 1. In some embodiments, the composition comprising nanoparticles comprising paclitaxel and albumin is administered qwx3 (days 1, 8, and 15) per 28 day cycle and the ABT-263 is administered on days 2, 3, and 4. In some embodiments, the composition comprising nanoparticles comprising paclitaxel and albumin is administered qwx3 (days 1, 8, and 15) per 28 day cycle and the ABT-263 is administered on days 2 and 4. In some embodiments, the composition comprising nanoparticles comprising paclitaxel and albumin is administered qwx3 (days 1, 8, and 15) per 28 day cycle and the ABT-263 is administered on days 2, 3, and 4. In some embodiments, the composition comprising nanoparticles comprising paclitaxel and albumin is administered q3w and the ABT-263 is administered on days 1 and 3. In some embodiments, the composition comprising nanoparticles comprising paclitaxel and albumin is administered qwx3 (days 1, 8, and 15) per 28 day cycle and the ABT-263 is administered on days 3 and 4. In some embodiments, the composition comprising nanoparticles comprising paclitaxel and albumin is administered qwx3 (days 1, 8, and 15) per 28 day cycle and the ABT-263 is administered on days 2 and 3. In some embodiments, the composition comprising nanoparticles comprising paclitaxel and albumin is administered qwx3 (days 1, 8, and 15) per 28 day cycle and the ABT-263 is administered on day 4.

In some embodiments, the composition comprising nanoparticles comprising paclitaxel and albumin is administered weekly in a four week cycle (qw (days 1, 8, 15, and 21) per 28 day cycle) and the ABT-263 is administered on days 1, 2, 3, and 4. In some embodiments, the composition comprising nanoparticles comprising paclitaxel and albumin is administered qw (days 1, 8, 15, and 21) per 28 day cycle and the ABT-263 is administered on days 1, 2, and 3. In some embodiments, the composition comprising nanoparticles comprising paclitaxel and albumin is administered qw (days 1, 8, 15, and 21) per 28 day cycle and the ABT-263 is administered on days 1 and 2. In some embodiments, the composition comprising nanoparticles comprising paclitaxel and albumin is administered qw (days 1, 8, 15, and 21) per 28 day cycle and the ABT-263 is administered on day 1. In some embodiments, the composition comprising nanoparticles comprising paclitaxel and albumin is administered qw (days 1, 8, 15, and 21) per 28 day cycle and the ABT-263 is administered on days 2, 3, and 4. In some embodiments, the composition comprising nanoparticles comprising paclitaxel and albumin is administered q3w and the ABT-263 is administered on days 1 and 3. In some embodiments, the composition comprising nanoparticles comprising paclitaxel and albumin is administered qw (days 1, 8, 15, and 21) per 28 day cycle and the ABT-263 is administered on days 2 and 4. In some embodiments, the composition comprising nanoparticles comprising paclitaxel and albumin is administered qw (days 1, 8, 15, and 21) per 28 day cycle and the ABT-263 is administered on day 2. In some embodiments, the composition comprising nanoparticles comprising paclitaxel and albumin is administered qw (days 1, 8, 15, and 21) per 28 day cycle and the ABT-263 is administered on days 3 and 4. In some embodiments, the composition comprising nanoparticles comprising paclitaxel and albumin is administered qw (days 1, 8, 15, and 21) per 28 day cycle and the ABT-263 is administered on days 2 and 3. In some embodiments, the composition comprising nanoparticles comprising paclitaxel and albumin is administered qw (days 1, 8, 15, and 21) per 28 day cycle and the ABT-263 is administered on day 4.

In some embodiments, the ABT-263 is administered with only the first dose of the composition comprising paclitaxel and an albumin per cycle. In some embodiments, the ABT-263 is administered with only the first and second dose of the composition comprising paclitaxel and an albumin per cycle. In some embodiments, the ABT-263 is administered with only the first, second, and third dose of the composition comprising paclitaxel and an albumin per cycle. In some embodiments, the ABT-263 is administered with only the second dose of the composition comprising paclitaxel and an albumin per cycle. In some embodiments, the ABT-263 is administered with only the third dose of the composition comprising paclitaxel and an albumin per cycle.

The nanoparticle composition (and the ABT-263) described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In one variation of the invention, nanoparticles (such as albumin nanoparticles) can be administered by any acceptable route including, but not limited to, orally, intramuscularly, transdermally, intravenously, through an inhaler or other air borne delivery systems and the like.

A combination of the administration configurations described herein can be used. The combination therapy methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit and/or delay the development of the disease.

As will be understood by those of ordinary skill in the art, the appropriate doses of ABT-263 will be approximately those already employed in clinical therapies wherein the ABT-263 are administered alone or in combination with other agents. Variation in dosage will likely occur depending on the condition being treated. As described above, in some embodiments, the ABT-263 may be administered at a reduced level.

As will be understood by those of ordinary skill in the art, although many of the exemplary dosing regimen discussed above pertain to paclitaxel, they are equally applicable to other taxanes discussed herein.

### ABT-263

ABT-263 (Navitoclax, also known as N-(4-(4-(2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide) is an orally available small molecule inhibitor of Bcl-2, Bcl-xL, and Bcl-w, with Ki of ≤0.5 nM, ≤1 nM and ≤1 nM, respectively. ABT-263 and has a molecular weight of 974.6 g/mol and has the formula:

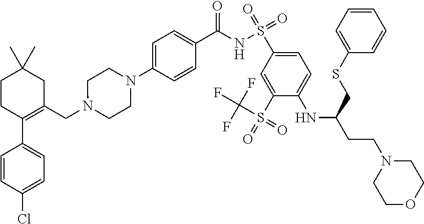 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

ABT-263 mimics a key portion of a BH3 α-helix that selectively targets Bch 2/Bcl-xL (Lee et al. (2007) Cell Death Differ. 14, 1711-1713; Petros et al. (2000) Protein Sci. 9: 2528-2534). Targeted inhibition of Bcl-2/Bcl-xL contributes to apoptosis induction in three ways: (1) blocking unoccupied Bcl-2/Bcl-xL pockets reduces the threshold for apoptosis—a sensitizing feature; (2) liberating sequestered BH3-only proteins enables them to occupy other antiapoptotic pockets and/or directly activate BAX/BAK; and (3) displacing the trapped forms of BAX/BAK frees their BH3 death helices to propel the homo-oligomerization process and consequent mitochondrial outer membrane permeabilization.

It is to be understood by one of ordinary skill in the art that the present invention encompasses various salt forms and crystalline polymorphic forms of ABT-263. Acid addition salts of ABT-263 include acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifuoroacetate, para-toluenesulfonate and undecanoate salts.

ABT-263 has at least two protonatable nitrogen atoms and is consequently capable of forming acid addition salts with more than one, for example about 1.2 to about 2, about 1.5 to about 2 or about 1.8 to about 2, equivalents of acid per equivalent of the compound. Illustratively, bis-salts can be formed including acetate, adipate, alginate, bicarbonate, citrate, aspartate, benzoate, besylate, bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate bis-salts, for example, bis-hydrochloride (bis-HCl) and bis-hydrobromide (bis-HBr) salts.

In some embodiments, the ABT-263 is ABT-263 bisHCl. ABT-263 bis-HCl, which has a molecular weight of 104 7.5 g/mol, is represented by the following structural formula:

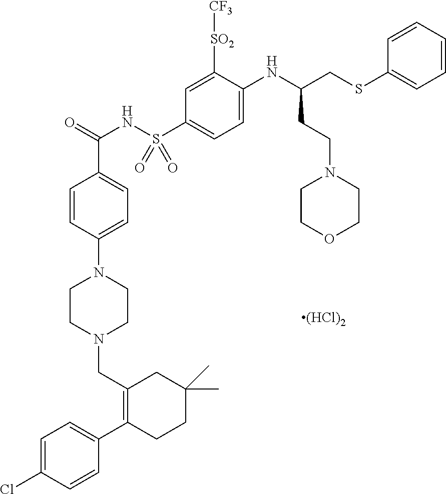 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

ABT-263 bis-HCl is also known as N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino-3-((trifluoromethyl)sulfonyl)benzenesulfonamide bis-hydrochloride

Solvated crystal forms of ABT-263 include ethanol, 1-propanol, 2-propanol, 2-butanol, t-butanol, nitromethane, acetonitrile, propionitrile, ethyl formate, methyl acetate, ethyl acetate, isopropyl acetate, acetone, 2-butanone (methyl ethyl ketone, MEK), methyl isopropyl ketone, 1,4-dioxane, benzene, toluene and butyl ether solvates.

Further details regarding ABT-263 are described in Tse et al. (2008) Cancer Res. 68: 3421-3428; Walensky (2011) J Clin Oncol. 30, 554-557; U.S. Pat. Nos. 8,362,013, 8,362,014, US 20110159085, and US 20100278921, which are incorporated herein by reference in their entireties. The present application specifically incorporate the ABT-263 described in these references by reference. In some embodiments, the ABT-263 is the crystalline polymorph Form I of ABT-263 bisHCl as described in U.S. Pat. No. 8,362,013. In some embodiments, the ABT-263 is the crystalline polymorph Form II of ABT-263 bisHCl as described in U.S. Pat. No. 8,362,013.

### Nanoparticle Compositions

The nanoparticle compositions described herein comprise nanoparticles comprising (in various embodiments consisting essentially of) a taxane (such as paclitaxel) and an albumin (such as human serum albumin). Nanoparticles of poorly water soluble drugs (such as taxane, e.g., paclitaxel) and methods of making thereof have been disclosed in, for example, U.S. Pat. Nos. 5,916,596; 6,506,405; 6,749,868, 6,537,579, and 7,820,788 and also in U.S. Pat. Pub. Nos. 2006/0263434, and 2007/0082838; PCT Patent Application WO08/137148, each of which is incorporated by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the albumin (such as human serum albumin) has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprise the taxane (such as paclitaxel) coated with an albumin, such as albumin (e.g., human serum albumin). In some embodiments, the composition comprises taxane (such as paclitaxel) in both nanoparticle and non-nanoparticle forms, wherein at least about any one of 50%, 60%, 70%, 80%, 90%, 95%, or 99% of the taxane in the composition are in nanoparticle form. In some embodiments, the taxane in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane that is substantially free of polymeric materials (such as polymeric matrix).

In some embodiments, the composition comprises albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the nanoparticle composition is substantially free (such as free) of surfactants (such as Cremophor®, Tween 80, or other organic solvents used for the administration of taxanes). In some embodiments, the nanoparticle composition contains less than about any one of 20%, 15%, 10%, 7.5%, 5%, 2.5%, or 1% organic solvent. In some embodiments, the weight ratio of albumin and taxane in the nanoparticle composition is about 18:1 or less, such as about 15:1 or less, for example about 10:1 or less. In some embodiments, the weight ratio of albumin (such as human serum albumin) and taxane (such as paclitaxel) in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of albumin and taxane (such as paclitaxel) in the nanoparticle portion of the composition is about any one of 1:2, 1:3, 1:4, 1:5, 1:10, 1:15, or less. In some embodiments, the weight ratio of the albumin (such as human serum albumin) and the taxane (such as paclitaxel) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the albumin is human serum albumin. Human serum albumin (HSA) is a highly soluble globular protein of Mr 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolemic shock (see, e.g., Tullis, JAMA, 4, 237, 355-360, 460-463, (1977)) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human serum albumin) in the composition generally serves as a carrier for the taxane (such as paclitaxel), i.e., the albumin in the composition makes the taxane more readily suspendable in an aqueous medium or helps maintain the suspension as compared to compositions not comprising an albumin. This can avoid the use of toxic solvents (or surfactants) for solubilizing the taxane, and thereby can reduce one or more side effects of administration of the taxane into an individual (such as a human). Thus, in some embodiments, the composition described herein is substantially free (such as free) of surfactants, such as Cremophor (including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the composition is not sufficient to cause one or more side effect(s) in an individual when the nanoparticle composition is administered to the individual. In some embodiments, the nanoparticle composition contains less than about any one of 20%, 15%, 10%, 7.5%, 5%, 2.5%, or 1% organic solvent or surfactant.

The amount of albumin (such as human serum albumin) in the composition described herein will vary depending on other components in the composition. In some embodiments, the composition comprises an albumin in an amount that is sufficient to stabilize the taxane in an aqueous suspension, for example, in the form of a stable colloidal suspension (such as a stable suspension of nanoparticles). In some embodiments, the albumin is in an amount that reduces the sedimentation rate of the taxane in an aqueous medium. For particle-containing compositions, the amount of the albumin also depends on the size and density of nanoparticles of the taxane.

A taxane, e.g., paclitaxel, is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin (such as human serum albumin) is present in an amount that is sufficient to stabilize the taxane in an aqueous suspension at a certain concentration. For example, the concentration of the taxane (such as paclitaxel) in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane (such as paclitaxel) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of albumin to the taxane in the nanoparticle composition is such that a sufficient amount of taxane binds to, or is transported by, the cell. While the weight ratio of albumin to taxane will have to be optimized for different albumin and taxane combinations, generally the weight ratio of albumin to taxane (such as paclitaxel) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to taxane weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human serum albumin) and the taxane (such as paclitaxel) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to an individual (such as human) without significant side effects. In some embodiments, the albumin is in an amount that is effective to reduce one or more side effects of administration of the taxane (such as paclitaxel) to a human. The term “reducing one or more side effects of administration of the taxane” refers to reduction, alleviation, elimination, or avoidance of one or more undesirable effects caused by the taxane, as well as side effects caused by delivery vehicles (such as solvents that render the taxanes suitable for injection) used to deliver the taxane (such as paclitaxel). Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxanes (such as paclitaxel) can be reduced.

In some embodiments, the nanoparticle compositions described herein comprises nanoparticles comprising a taxane (such as paclitaxel) and an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) and an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) and an albumin (such as human albumin or human serum albumin), wherein the nanoparticles have an average diameter of about 130 nm. In some embodiments, the nanoparticle compositions described herein comprises nanoparticles comprising paclitaxel and human albumin (such as human serum albumin), wherein the nanoparticles have an average diameter of about 130 nm.

In some embodiments, the nanoparticle compositions described herein comprises nanoparticles comprising a taxane (such as paclitaxel) and an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) and an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) and an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel and human albumin (such as human serum albumin), wherein the nanoparticles have an average diameter of about 130 nm, wherein the weight ratio of albumin and the taxane in the composition is about 9:1.

In some embodiments, the nanoparticle compositions described herein comprises nanoparticles comprising a taxane (such as paclitaxel) coated with an albumin (such as human albumin or human serum albumin). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) coated with an albumin (such as human albumin or human serum albumin), wherein the nanoparticles have an average diameter of about 130 nm. In some embodiments, the nanoparticle compositions described herein comprises nanoparticles comprising paclitaxel coated with human albumin (such as human serum albumin), wherein the nanoparticles have an average diameter of about 130 nm.

In some embodiments, the nanoparticle compositions described herein comprises nanoparticles comprising a taxane (such as paclitaxel) coated with an albumin (such as human albumin or human serum albumin), wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) coated with an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel coated with human albumin (such as human serum albumin), wherein the nanoparticles have an average diameter of about 130 nm, wherein the weight ratio of albumin and the taxane in the composition is about 9:1.

In some embodiments, the nanoparticle compositions described herein comprises nanoparticles comprising a taxane (such as paclitaxel) stabilized by an albumin (such as human albumin or human serum albumin). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 200 nm. In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 150 nm. In some embodiments, the nanoparticle compositions described herein comprises nanoparticles comprising a taxane (such as paclitaxel) stabilized by an albumin (such as human albumin or human serum albumin), wherein the nanoparticles have an average diameter of about 130 nm. In some embodiments, the nanoparticle compositions described herein comprises nanoparticles comprising paclitaxel stabilized by human albumin (such as human serum albumin), wherein the nanoparticles have an average diameter of about 130 nm.

In some embodiments, the nanoparticle compositions described herein comprises nanoparticles comprising a taxane (such as paclitaxel) stabilized by an albumin (such as human albumin or human serum albumin), wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 200 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of no greater than about 150 nm, wherein the weight ratio of the albumin and the taxane in the composition is no greater than about 9:1 (such as about 9:1). In some embodiments, the nanoparticle compositions described herein comprises nanoparticles comprising a taxane (such as paclitaxel) stabilized by an albumin (such as human albumin or human serum albumin), wherein the nanoparticles have an average diameter of about 150 nm, wherein the weight ratio of the albumin and the taxane in the composition is no greater than about 9:1 (such as about 9:1). In some embodiments, the nanoparticle compositions described herein comprises nanoparticles comprising paclitaxel stabilized by human albumin (such as human serum albumin), wherein the nanoparticles have an average diameter of about 130 nm, wherein the weight ratio of albumin and the taxane in the composition is about 9:1.

In some embodiments, the composition comprises nanoparticle albumin bound paclitaxel, nab-paclitaxel (such as Abraxane). Abraxane® is a formulation of paclitaxel stabilized by human albumin USP, which can be dispersed in directly injectable physiological solution. When dispersed in a suitable aqueous medium such as 0.9% sodium chloride injection or 5% dextrose injection, Abraxane® forms a stable colloidal suspension of paclitaxel. The weight ratio of human albumin and paclitaxel in the composition is about 9:1. The mean particle size of the nanoparticles in the colloidal suspension is about 130 nanometers. Since HSA is freely soluble in water, Abraxane® can be reconstituted in a wide range of concentrations ranging from dilute (0.1 mg/ml paclitaxel) to concentrated (20 mg/ml paclitaxel), including for example about 2 mg/ml to about 8 mg/ml, about 5 mg/ml.

Methods of making nanoparticle albumin bound nanoparticle compositions are known in the art. For example, nanoparticles containing taxanes (such as paclitaxel) and albumin (such as human serum albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579, 7,820,788 and also in U.S. Pat. Pub. No. 2007/0082838, 2006/0263434 and PCT Application WO08/137148.

Briefly, the taxane (such as paclitaxel) is dissolved in an organic solvent, and the solution can be added to a human serum albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1.

### Other Components in the Nanoparticle Compositions

The nanoparticles described herein can be present in a composition that include ABT-263s, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the nanoparticle composition (see, e.g., U.S. Pat. Nos. 5,916,596, 6,096,331 and 7,820,788). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Kits, Medicines, and Compositions

The invention also provides compositions (such as pharmaceutical compositions), medicine, kits, and unit dosages useful for methods described herein. Also provided are any use described herein whether in the context of use as a medicament and/or use for manufacture of a medicament.

Kits of the invention include one or more containers comprising taxane-containing nanoparticle compositions (or unit dosage forms and/or articles of manufacture) and/or ABT-263, and in some embodiments, further comprise instructions for use in accordance with any of the methods described herein. The kit may further comprise a description of selection an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising nanoparticles comprising a taxane (such as paclitaxel) and an albumin, and b) ABT-263. In some embodiments, the kit comprises a) a composition comprising nanoparticles comprising a taxane (such as paclitaxel) and an albumin, b) ABT-263, and c) instructions for administering the nanoparticles and the ABT-263s simultaneously, sequentially, or concurrently for treatment of cancer. In some embodiments, the taxane is any of paclitaxel, docetaxel, and ortataxel. In some embodiments, the kit comprises nanoparticles comprising a) a composition comprising nanoparticles comprising paclitaxel coated with an albumin (such as nab-paclitaxel for example Abraxane), b) an effective amount of ABT-263, and c) instructions for administering the nanoparticles and the ABT-263 simultaneously, sequentially, and/or concurrently, for the effective treatment of cancer.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the nanoparticle compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the taxane (such as paclitaxel) as disclosed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the taxane and pharmaceutical compositions and instructions for use and packaged in quantities sufficient for storage and use in pharmacies, for example, hospital pharmacies and compounding pharmacies.

Also provided are medicines for treating proliferative diseases. In some embodiments, the medicine comprises a) a composition comprising nanoparticles comprising a taxane (such as paclitaxel) and an albumin, and b) ABT-263. In some embodiments, the taxane is any of paclitaxel, docetaxel, and ortataxel. In some embodiments, the kit comprises nanoparticles comprising a) a composition comprising nanoparticles comprising paclitaxel coated with an albumin (such as nab-paclitaxel for example Abraxane®), and b) ABT-263, and c) instructions for administering the nanoparticles and the ABT-263s simultaneously, sequentially, and/or concurrently, for the effective treatment of cancer.

In some embodiments, the medicine comprises a) a composition comprising nanoparticles comprising a taxane (such as paclitaxel) and an albumin, and b) ABT-263. In some embodiments, the taxane is any of paclitaxel, docetaxel, and ortataxel.

Also provided are medicines for treating lung cancer. In some embodiments, the medicine comprises a) a composition comprising nanoparticles comprising a taxane (such as paclitaxel) and an albumin, and b) ABT-263. In some embodiments there is provided a kit comprising nanoparticles comprising a) a composition comprising nanoparticles comprising paclitaxel coated with an albumin (such as nab-paclitaxel for example Abraxane), and b) ABT-263, and c) instructions for administering the nanoparticles and the ABT-263 simultaneously, sequentially, and/or concurrently, for the effective treatment of lung cancer.

Also provided are medicines for treating pancreatic cancer. In some embodiments, the medicine comprises a) a composition comprising nanoparticles comprising a taxane (such as paclitaxel) and an albumin, and b) ABT-263. In some embodiments, the taxane is any of paclitaxel, docetaxel, and ortataxel. In some embodiments there is provided a kit comprising nanoparticles comprising a) a composition comprising nanoparticles comprising paclitaxel coated with an albumin (such as nab-paclitaxel for example Abraxane), and b) ABT-263, and c) instructions for administering the nanoparticles and the ABT-263 simultaneously, sequentially, and/or concurrently, for the effective treatment of pancreatic cancer. In some embodiments there is provided a kit comprising nanoparticles comprising a) a composition comprising nanoparticles comprising paclitaxel coated with an albumin (such as nab-paclitaxel for example Abraxane®), and b) ABT-263, and c) instructions for administering the nanoparticles and the ABT-263 simultaneously, sequentially, and/or concurrently, for the effective treatment of pancreatic cancer.

Also provided are medicines for treating breast cancer (for example, HER2 negative breast cancer or for example, triple negative breast cancer). In some embodiments, the medicine comprises a) a composition comprising nanoparticles comprising a taxane (such as paclitaxel) and an albumin, and b) ABT-263. In some embodiments, the taxane is any of paclitaxel, docetaxel, and ortataxel. In some embodiments there is provided a kit comprising nanoparticles comprising a) a composition comprising nanoparticles comprising paclitaxel coated with an albumin (such as nab-paclitaxel for example Abraxane), and b) ABT-263, and c) instructions for administering the nanoparticles and the ABT-263 simultaneously, sequentially, and/or concurrently, for the effective treatment of breast cancer (for example, HER2 negative breast cancer or for example, triple negative breast cancer).

The nanoparticles and the ABT-263 can be present in separate containers or in a single container. It is understood that the medicine may comprise one distinct composition or two or more compositions wherein one composition comprises nanoparticles and one composition comprises anABT-263.

The kits, medicines, and compositions of this invention may include any one or more aspects or parameters described herein.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1. Effect of Nab-Paclitaxel (ABRAXANE®) in Combination with ABT-263 on Xenografted MiaPACA2 Tumors

Microinjection experiments were performed to assess the efficacy of (a) nab-paclitaxel (ABRAXANE®), (b) ABT-199 (an inhibitor of Bcl-2), (c) ABT-263(an inhibitor of Bcl-2 and Bcl-xL), (d) nab-paclitaxel in combination with ABT-199, and (e) nab-paclitaxel in combination with ABT-263 in mice bearing human pancreatic carcinoma MiaPACA2 xenograft tumors.

Microinjections were performed using the CIVO™ arrayed microinjection device (Presage Biosciences, Seattle Wash.). This device enables simultaneous delivery of multiple drug samples, each individually, directly into spatially defined positions within a living tumor. The CIVO device having a 5 needle array was inserted transcutaneously into flank tumors of anesthetized mice.

Tumors were injected with either nab-paclitaxel, ABT-263, nab-paclitaxel in combination with ABT-263, ABT-199, or nab-paclitaxel in combination with ABT-199. 24 hours later, tumors were resected and sectioned across the axis of injection to produce histological sections of tumor that show the 5 different sites of injection. The sections were then processed for immunofluorescence analysis with DAPI (a stain that binds strongly to AT-rich regions in DNA), antibodies against cleaved caspase-3 (CC3, an apoptosis marker), IFN stimulatory DNA (ISD, a ligand that binds non-AT-rich regions in DNA) and antibodies against phophohistone-H3 (pHH3, a marker of cells undergoing mitosis).

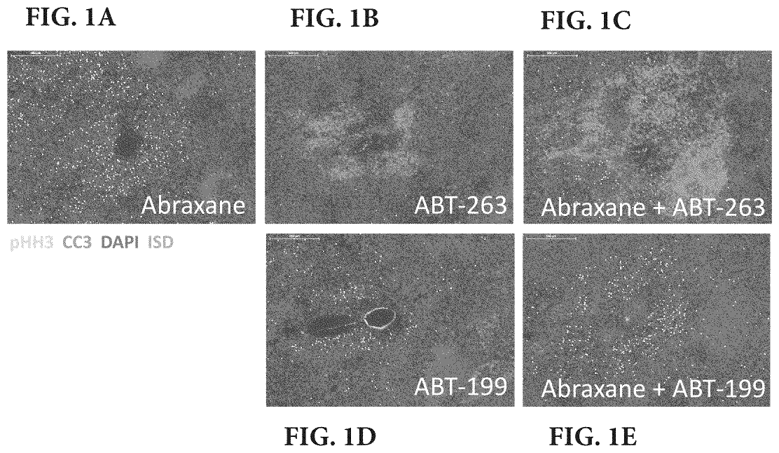

Representative images from a single injection site are shown in FIG. 1. MiaPACA2 tumors injected with nab-paclitaxel and ABT-263 (FIG. 1C) exhibited higher CC3+staining than tumors injected with nab-paclitaxel alone (FIG. 1A), ABT-263 alone (FIG. 1B), ABT-199 alone (FIG. 1D), or nab-paclitaxel in combination with ABT-199 (FIG. 1E).

Circular regions of interests (ROI) centered on the injection sites were inscribed. Within each ROI, the fraction of cells expressing CC3 was mapped as a function of radial distance from the injection site. To generate radial response plots, calculations were made at 100 micron intervals and were based on all cells within a 100 micron wide band centered at each radial distance. Measurements began at 200 microns to exclude regions directly affected by needle induced injury.

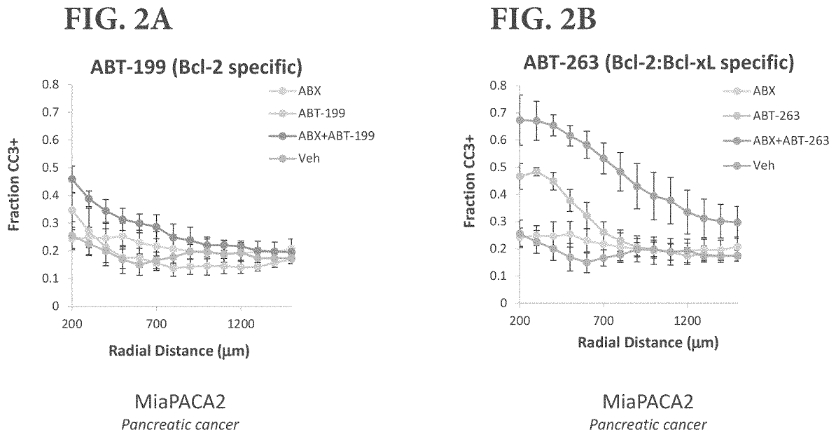

Injection with ABT-263, ABT-199, nab-paclitaxel, nab-paclitaxel/ABT-263, and nab-paclitaxel/ABT-199 induced an increase in CC3 positive cells at the site of injection that diminished with increasing radial distance indicating drug diffusion and/or clearance limited tumor cell exposure. The area of response and the total fraction of CC3 positive cells were significantly greater for tumors injected with nab-paclitaxel/ABT-263 (FIG. 2B) compared to ABT-263 alone (FIG. 2B), ABT-199 alone (FIG. 2A), nab-paclitaxel alone (FIGS. 2A-B), nab-paclitaxel/ABT-199 (FIG. 2A) or vehicle (FIGS. 2A-B).

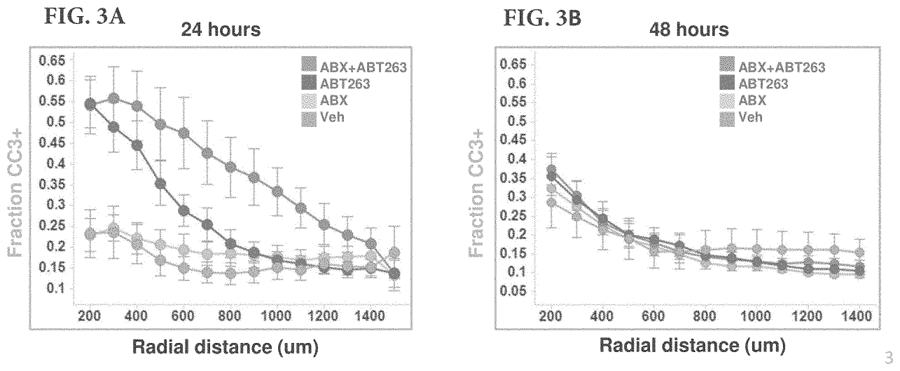

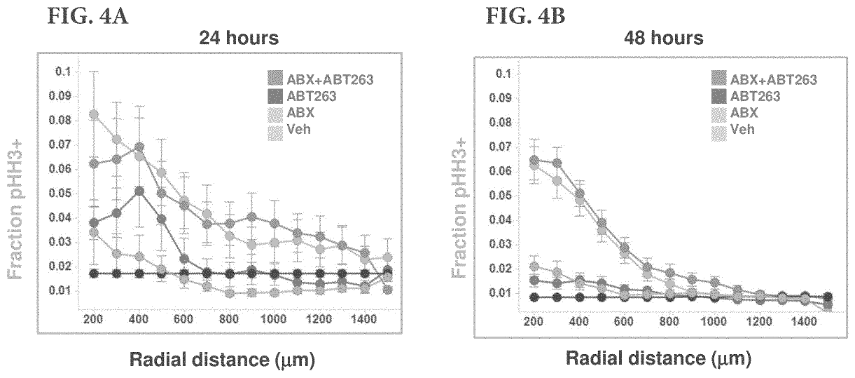

The experiments were repeated in mice bearing xenografted MiaPACA2 tumors using ABT-263, nab-paclitaxel, and nab-paclitaxel/ABT-263. Tumors from a first set of mice were processed for immunofluorescence analysis with CC3 antibodies 24 hours after injection (FIG. 3A), and tumors from a second set were processed for analysis with CC3 antibodies 48 hours after injection (FIG. 3B). Tumors from a third set of mice were processed for immunofluorescence analysis with pHH3 antibodies 24 hours after injection (FIG. 4A), and tumors from a fourth set were processed for analysis with pHH3 antibodies 48 hours after injection (FIG. 4B). Induction of apoptosis is transient and extends beyond 1 mm from the site of injection (FIGS. 3A and 3B). As shown in FIGS. 4A and 4B, ABT-263 does not have a detectable effect on the durability of nab-paclitaxel-induced mitotic arrest.

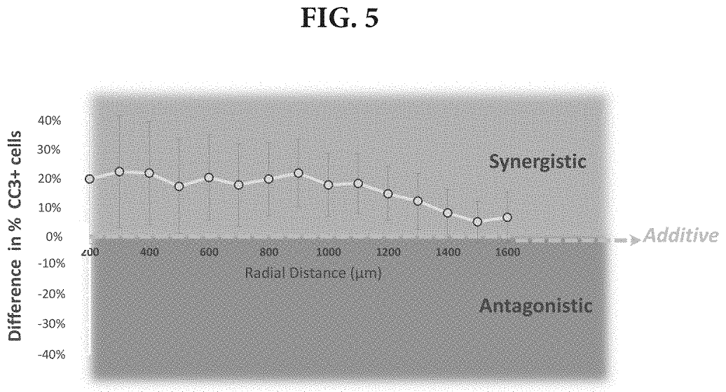

Mean values of percent CC3 positive cells were plotted with standard error bars, as a function of radial distance for each formulation and time point. To assess the statistical significance of differences between any pair of formulations, a linear mixed model approach was used. A p-value less than 0.05, adjusted for multiple comparisons, was taken to indicate statistically significant differences between any two comparisons. Such statistical modeling demonstrates that nab-paclitaxel/ABT-263 shows significant synergy compared to nab-paclitaxel alone or ABT-263 alone. See FIG. 5 (error bars indicate 95% confidence intervals).

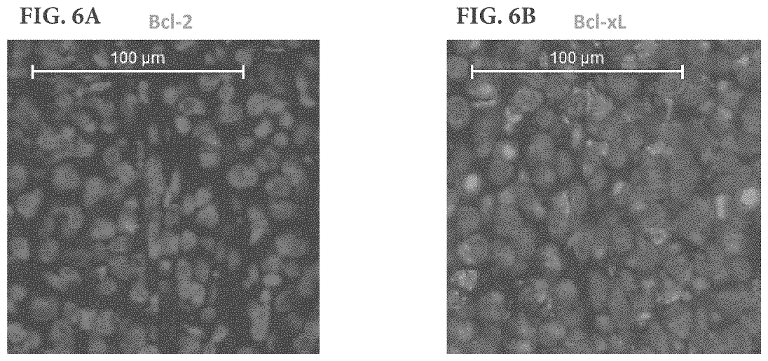

MiaPACA2 tumors were processed for immunofluorescence analysis with anti-Bcl-2 antibodies or anti-Bcl-xL antibodies. As shown in FIG. 6, Bcl-xL (FIG. 6B) is more highly expressed than Bcl-2 (FIG. 6A) in MiaPACA2 cells.

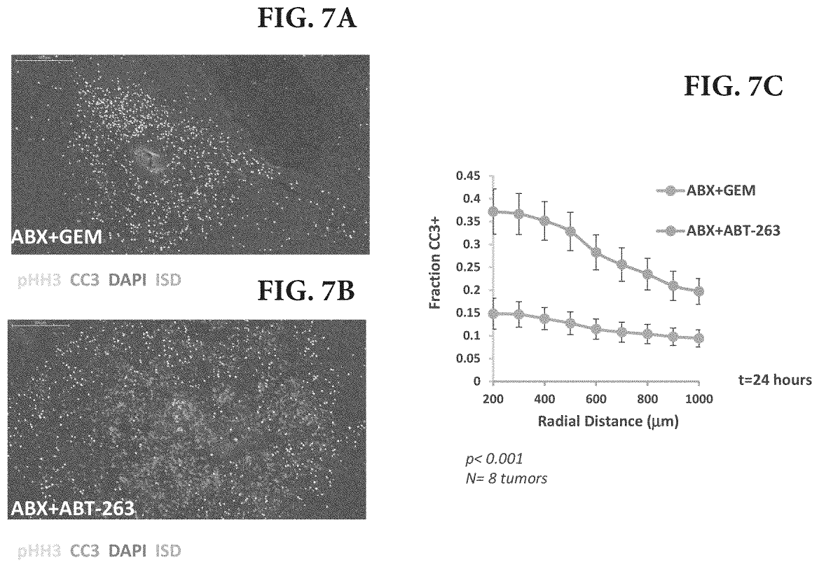

Further experiments were performed to compare the effects of nab-paclitaxel/ABT-263 to those of nab-paclitaxel/gemcitabine (i.e., the standard of care treatment for pancreatic cancer) in mice bearing xenografted MiaPACA2 tumors. Tumors were injected with nab-paclitaxel/ABT-263 or nab-paclitaxel/gemcitabine. After 24 hours, the tumors were processed for immunofluorescence analysis with CC3 antibodies. Representative images from a single injection site are shown for nab-paclitaxel/gemcitabine (FIG. 7A) and for nab-paclitaxel/ABT-263 (FIG. 7B). As shown in FIG. 7C, the area of response and the total fraction of CC3 positive cells were significantly greater for tumors injected with nab-paclitaxel/ABT-263 (FIG. 7B) compared to tumors injected with nab-paclitaxel/gemcitabine (FIG. 7A).

### Example 2. Effect of Nab-Paclitaxel (ABRAXANE®) in Combination with ABT-263 on Xenografted H2122 Tumors

Experiments were performed to assess the efficacy of (a) nab-paclitaxel (ABRAXANE 0), (b) ABT-263 (an inhibitor of Bcl-2 and Bcl-xL), and (e) nab-paclitaxel in combination with ABT-263 in mice bearing human non small cell lung cancer (NSCLC) H2122 xenograft tumors using the CIVO™ arrayed microinjection device as described above.

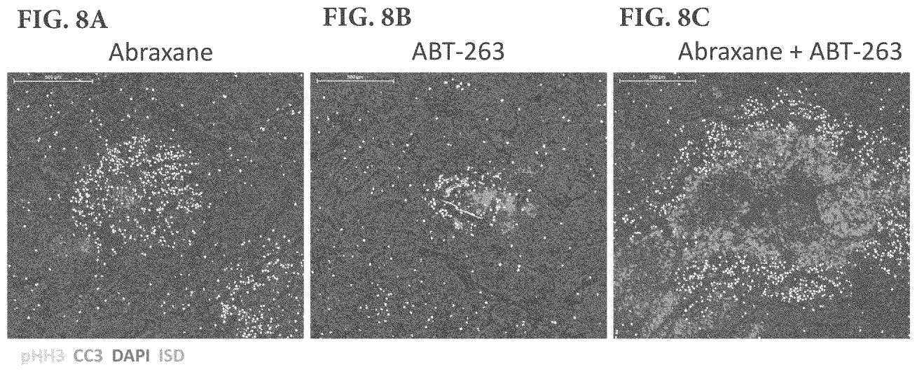

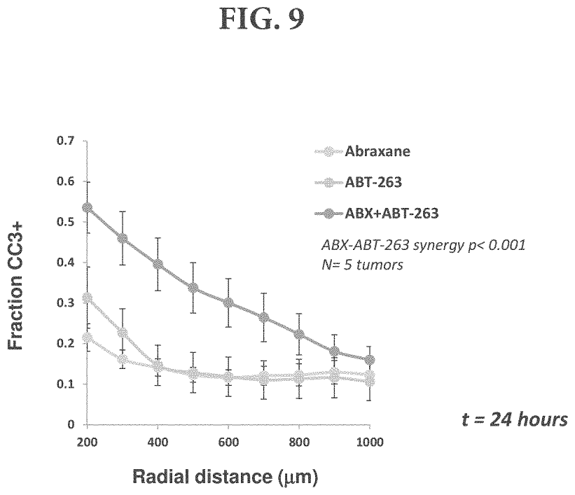

Tumors were injected with either nab-paclitaxel, ABT-263, nab-paclitaxel in combination with ABT-263. The tumors were then processed as described above for immunofluorescence analysis with DAPI, antibodies against CC3, IFN stimulatory DNA (ISD) and antibodies against phophohistone-H3 (pHH3). H2122 tumors injected with nab-paclitaxel and ABT-263 (FIG. 8C) exhibited higher CC3+staining than tumors injected with nab-paclitaxel alone (FIG. 8A) or ABT-263 alone (FIG. 8B). The area of response and the total fraction of CC3 positive cells were significantly greater for tumors injected with nab-paclitaxel/ABT-263 compared to ABT-263 alone or nab-paclitaxel alone (FIG. 9).

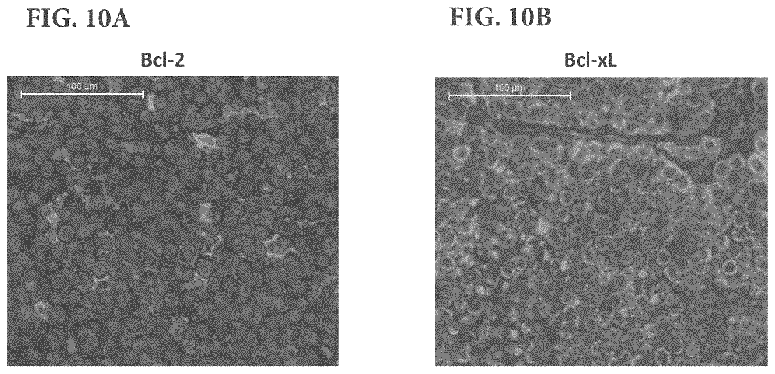

H2122 tumors were processed for immunofluorescence analysis with anti-Bch 2 antibodies or anti-Bcl-xL antibodies. As shown in FIG. 10, Bcl-xL (FIG. 10B) is more highly expressed than Bcl-2 (FIG. 10A) in H2122 cells.

1. A method of treating a cancer in an individual, comprising administering to the individual a) an effective amount of a composition comprising nanoparticles comprising a taxane and an albumin, and b) an effective amount of ABT-263.

2. The method of claim 1, wherein the nanoparticles in the composition are coated with albumin.

3. The method of claim 1, wherein the taxane is paclitaxel.

4. The method of claim 1, wherein the nanoparticle composition is administered intravenously.

5. The method of claim 1, wherein the nanoparticle composition is administered at the dosage range of about 60 to about 300 mg/m².

6. The method of claim 1, wherein the ABT-263 is administered orally.

7. The method of claim 1, wherein the ABT-263 is administered at the dosage range of about 10 to about 300 mg/day.

8. The method of claim 1, wherein the composition comprising nanoparticles and the ABT-263 are administered simultaneously or sequentially.

9-11. (canceled)

12. The method of claim 1, wherein the cancer is:
(a) resistant or refractory to the treatment of taxane when administered alone or in conjunction with an agent other than ABT-263, or
(b) resistant or refractory to the treatment of ABT-263 when administered alone or in conjunction with an agent other than the nanoparticle composition.

13. (canceled)

14. The method of claim 1, wherein the cancer is selected from the group consisting of lung cancer, pancreatic cancer, breast cancer, gastric cancer, or colorectal cancer.

15. (canceled)

16. The method of claim 14, wherein the cancer is non-small cell lung cancer (NSCLC), metastatic pancreatic cancer, or locally advanced pancreatic cancer.

17. The method of claim 14, wherein the method further comprises administering to the individual an effective amount of carboplatin or gemcitabine.

18. The method of claim 17, wherein the method further comprises administering to the individual an effective amount of carboplatin, and wherein the carboplatin is administered at the dosage of AUC=2 to AUC=6.

19-21. (canceled)

22. The method of claim 17, wherein the method further comprises administering to the individual an effective amount of gemcitabine, and wherein the gemcitabine is administered at the dosage of about 1000 to about 2000 mg/m².

23. (canceled)

24. The method of claim 14, wherein the cancer is breast cancer,
and wherein the individual is negative for ER, PR, and HER2.

25. The method of claim 24, wherein the cancer is breast cancer, and wherein the method further comprises conducting definitive surgery within about 1 to about 10 days following the treatment.

26. The method of claim 1, wherein the cancer is squamous cell carcinoma or adenocarcinoma.

27. (canceled)

28. The method of claim 1, wherein the individual overexpresses Bcl-xL.

29. The method of claim 28, wherein the individual does not overexpress Bcl-2.

30. The method of claim 1, wherein the nanoparticles in the composition have an average diameter of no greater than about 200 nm.

31. The method of claim 1, wherein the weight ratio of albumin and taxane in the nanoparticle composition is about 18:1 or less.

32. The method of claim 1, wherein the ABT-263 is ABT-263 bisHCl.

33. A kit for treating cancer in an individual, comprising a) an effective amount of a composition comprising nanoparticles comprising a taxane and an albumin, b) an effective amount of ABT-263, and c) instructions for use.
